(19) United States
(12) Patent Application Publication
(10) Pub. No.: US 2013/0023428 A1
(43) Pub. Date: Jan. 24, 2013

(54) BIOMARKERS FOR NEUROLOGICAL CONDITIONS

(71) Applicants: Claudius Mueller, Gainesville, VA (US); Matthew Schrag, Grand Terrace, CA (US); Lance Liotta, Bethesda, MD (US); Wolff M. Kirsch, Redlands, CA (US); Harry V. Vinters, Venice, CA (US)

(72) Inventors: Claudius Mueller, Gainesville, VA (US); Matthew Schrag, Grand Terrace, CA (US); Lance Liotta, Bethesda, MD (US); Wolff M. Kirsch, Redlands, CA (US); Harry V. Vinters, Venice, CA (US)

(21) Appl. No.: 13/393,817

(22) PCT Filed: Sep. 2, 2010
(86) PCT No.: PCT/US10/47751

§ 371 Date: Sep. 27, 2012

Related U.S. Application Data
(60) Provisional application No. 61/239,732, filed on Sep. 3, 2009.

(51) Int. Cl. C40B 30/04; C40B 40/10; G01N 33/573; G01N 33/566; G01N 27/62

(52) U.S. Cl. 506/9; 435/7.92; 435/23; 435/7.4; 435/7.21; 506/18

(57) ABSTRACT

Methods and kits for identifying and/or monitoring neurological conditions in a patient using ratios of biomarkers are disclosed. The neurological conditions may include, for example, Alzheimer's disease or mild cognitive impairment. The particular biomarkers that may be useful in identifying and/or monitoring neurological conditions may include, for example, biliverdin reductase, biliverdin reductase, estrogen receptor alpha, superoxide dismutase, S100A7, hemeoxygenase 1, matrix metalloproteinase 9 and platelet derived growth factor receptor. In particular, ratios of these biomarkers are useful.

### BACKGROUND

1. Field of the Disclosure

The described technology relates to the fields of molecular biology and medicine. In particular, disclosed herein are methods for diagnosing neurological conditions in a patient by using ratios of selected biomarkers.

2. Description of the Related Technology

Alzheimer's disease (AD) is a progressive degenerative disease of the brain primarily associated with aging. AD is one of several disorders that cause the gradual loss of brain cells and is a leading cause of dementia. Clinical presentation of AD is characterized by loss of memory, cognition, reasoning, judgment, and orientation. Mild cognitive impairment (MCI) is often the first identified stage of AD. As the disease progresses, motor, sensory, and linguistic abilities also are affected until there is global impairment of multiple cognitive functions. These cognitive losses occur gradually, but typically lead to severe impairment and eventual death in the range of three to twenty years.

An early diagnosis of AD has many advantages including, for example, increased time to maximize quality of life, reduced anxiety about unknown problems, increased chances of benefiting from treatment and increased time to plan for the future. However, reliable and noninvasive methods for diagnosing AD are not available.

Alzheimer's disease is characterized by two major pathologic observations in the brain: neurofibrillary tangles (NFT) and beta-amyloid plaques, comprised predominantly of an aggregate of fragments known as Aβ peptides. Individuals with AD exhibit characteristic beta-amyloid deposits in the brain (beta-amyloid plaques) and in cerebral blood vessels (beta-amyloid angiopathy) as well as neurofibrillary tangles. Neurofibrillary tangles occur not only in Alzheimer's disease but also in other dementia-inducing disorders. On autopsy, presently the only definitive method of diagnosing AD, large numbers of these lesions are generally found in areas of the human brain important for memory and cognition.

While advances have been made in imaging beta-amyloid, (Lopresti et al. J. Nuel. Med. (2005) 46:1959-1972), no serum biomarkers for AD are clinically available that can detect early stage AD, particularly at the stage of MCI. There are no validated biomarkers for confirming the diagnosis of a major neurodegenerative disorder or to monitor progression (Castano et al. Neurol. Res. (2006) 28:1155-163).

Despite the enthusiasm for the use of proteomic technology to discover blood markers of AD and decades of effort, progress towards identifying useful markers has been slow. The slow progress may have been because putative high specificity AD markers have been assumed to be in very low abundance because they are shed from small volumes of diseased tissue and are expected to be rapidly cleared and metabolized. In addition, researchers have avoided studying blood because the blood proteome is complicated by, resident proteins such as albumin that can exist at a concentration many millions of times greater than the target low abundance biomarker. For this reason, researchers have focused on cerebrospinal fluid (CSF) as the target fluid for AD biomarkers (see Zhang et al., J. Alzheimer's Disease (2005) 8:377-3386). The CSF approach, however, has limited clinical application to routine screening. Moreover, the blood brain vascular circulation perfuses AD lesions with a higher efficiency, particularly in the case for amyloid angiopathy.

### SUMMARY OF CERTAIN INVENTIVE ASPECTS

In one aspect, a method for diagnosing a neurological condition in a subject is provided. The method may include, for example, obtaining a biological sample from a subject suspected of being at risk for said neurological condition; determining a level of expression of at least one first biomarker in said biological sample from said subject; determining a level of expression of at least one second biomarker in said biological sample from said subject; and determining a ratio of said first biomarker to said second biomarker; and comparing the level of the ratio to a predetermined level, thereby diagnosing said neurological condition in said subject. In some embodiments, a difference in said ratio compared to the predetermined level indicates said neurological condition. In some embodiments, a method for diagnosing a neurological condition includes identifying a subject suspected of being at risk for said neurological condition.

In another aspect, a method for monitoring the progress of a neurological condition in a subject is provided. The method may include, for example, obtaining a first biological sample from a subject with said neurological condition at a first time; obtaining a second biological sample from said subject at a second time; determining a level of expression of at least one first biomarker in said first biological sample and said second biological sample; determining a level of expression of at least one second biomarker in said first biological sample and said second biological sample; determining a first ratio of said first biomarker to said second biomarker in said first biological sample; determining a second ratio of said first biomarker to said second biomarker in said second biological sample; and comparing the level of the first ratio and the second ratio, thereby monitoring the progress of said neurological condition in said subject. In some embodiments, a difference in said first ratio compared to said second ratio indicates the progress of said neurological condition. In some embodiments, a method for monitoring the progress of a neurological condition in a subject further includes identifying a subject with said neurological condition.

In another aspect, a kit is provided. In some embodiments, the kit includes, for example, a first agent that specifically detects at least one first biomarker; a second agent that specifically detects at least one second biomarker; and instructions for using the kit components to determine the level of expression of said first biomarker and said second biomarker and to determine a ratio of said first biomarker to said second biomarker in a person at risk for a neurological condition. In some embodiments, the first agent that specifically detects said first biomarker is an antibody that binds to said first biomarker. In some embodiments, the second agent that specifically detects said second biomarker is an antibody that binds to said second biomarker.

In some embodiments, the first biomarker is selected from the group including biliverdin reductase (BLVR), biliverdin reductase B (BLVRB), estrogen receptor alpha (ERA), S100A7, hemeoxygenase 1 (HO1), matrix metalloproteinase 9 (MMP9) and platelet derived growth factor receptor beta (PDGFR). In some embodiments, the second biomarker is selected from the group including BLVR, BLVRB, ERA, S100A7, HO1, MMP9, and PDGFR. In some embodiments, the first biomarker includes ERA and said second biomarker includes BLVR. In some embodiments, the first biomarker includes MMP9 and said second biomarker includes BLVR. In some embodiments, the first biomarker includes BLVRB and said second biomarker includes BLVR. In some embodiments, the first biomarker includes HO1 and said second biomarker includes BLVR. In some embodiments, the first biomarker includes PDGFR and said second biomarker includes BLVR. In some embodiments, the first biomarker includes S100A7 and said second biomarker includes BLVR. In some embodiments, the first biomarker includes ERA and said second biomarker includes BLVRB. In some embodiments, the first biomarker includes HO1 and said second biomarker includes BLVRB. In some embodiments, the first biomarker includes MMP9 and said second biomarker includes HO1. In some embodiments, the first biomarker includes PDGFR and said second biomarker includes HO1. In some embodiments, the first biomarker includes S100A7 and said second biomarker includes ERA.

In some embodiments, the biological sample includes blood, serum or plasma.

In some embodiments, determining the level of expression of the first and second biomarkers includes, for example, determining the level of mRNA for the first and second biomarkers. In some embodiments, determining the level of expression of the first and second biomarkers includes determining the level of protein for the first and second biomarkers. In some embodiments, determining the level of expression of the first and second biomarkers includes contacting said biological sample with antibodies against the first and second biomarkers. In some embodiments, determining the level of expression of the first and second biomarkers includes an assay selected from the group including immunoassay, mass spectrometry, immuno-mass spectrometry and suspension bead array. In some embodiments, the immunoassay includes an enzyme linked immunosorbent assay (ELISA). In some embodiments, the mass spectrometry includes tandem mass spectroscopy (MSMS).

In some embodiments, the method further includes obtaining a neuroimage of brain microvasculopathy. In some embodiments, the neuroimage is obtained by a method selected from the group including susceptibility weighted imaging and magnetic resonance spectroscopy.

In some embodiments, the neurological condition is selected from the group including Alzheimer's disease, mild cognitive impairment, stable mild cognitive impairment, mild Alzheimer's disease, vascular dementia, angiopathy black holes, cerebral amyloid angiopathy, and microhemorrages. In some embodiments, the neurological condition is Alzheimer's disease. In some embodiments, the neurological condition is mild cognitive impairment. In some embodiments, the neurodegenerative disease is microhemorrages.

### BRIEF DESCRIPTION OF THE DRAWINGS

Aspects of the disclosure will be readily apparent from the description below and the appended drawings, which are meant to illustrate and not to limit the disclosure, and in which:

FIG. 1 illustrates a flowchart of an experimental setup. Three approaches were used during the discovery phase of the project: using whole serum analyzed by disease group, low molecular weight (LMW) serum by disease group and LMW serum in the same patients before and after cognitive decline. Samples were analyzed using LC/MS-MS. During the validation phase abundance of selected biomarker candidates was measured in LMW serum using reverse phase protein arrays.

FIG. 2 illustrates functional protein classes (GO terms) of proteins/peptides. Only potential biomarker candidates are included that had a different spectral count after LC/MS-MS analysis between control, MCI and mild AD sera. (n for control, MCI and mild AD samples: whole serum: n=7, 5, 12; low molecular weight (LMW) serum by group: n=14, 14, 15. n for before and after cognitive decline: LMW serum longitudinal: n=3, 3.)

FIG. 3 illustrates ratios of staining intensities. Low molecular weight serum samples were analyzed using reverse phase protein arrays. Intensities were normalized against beta globin staining. FIG. 3A shows same patient samples before (extraction 1) and after significant cognitive decline (extraction 2). FIG. 3B shows samples of stable MCI patients (stable) versus cognitively declining MCI patients (decline), before cognitive decline in the second group. FIG. 3C shows samples of stable MCI patients (stable) versus cognitively declining MCI patients (decline), after cognitive decline in the second group (about 2 years later).

FIG. 4 illustrates ratios of staining intensities. Low molecular weight serum samples were analyzed using reverse phase protein arrays. Intensities were normalized against beta globin staining. Samples were analyzed by sample group.

FIG. 5 illustrates that the expression of heme degradation pathway components in AD plasma/serum is different from brain. Expression of HO-1 is upregulated in AD brain (Smith, et al. (1994) Am. J. Pathol. 145:42-47), most likely due to increased oxidative stress. Although the expression of BLVR has not been investigated in AD it can be upregulated by oxidative stress as well (Salim et al. (2001) J. Biol. Chem. 276:10929-10934). This is supported by the increase of bilirubin in AD cerebrospinal fluid (Kimpara et al. (2000) Neurobiol Aging 21:551-4). In AD plasma or serum the opposite happens. HO-1 is down-regulated (Schipper, et al. (2000) Neurology 54:1297-1304), probably through the action of upregulated α1-antitrypsin (Maes et al. (2006) Neurobiol Dis 24:89-100). As described herein, BLVR is also downregulated compared to HO-1 and other proteins in AD serum. This is further supported by the observation that levels of bilirubin are reduced in AD plasma (Kim et al. (2006) Int J Geriatr Psychiatry 21:344-8). (Figure legend: italicized text=level/expression not known; enzymatic reaction=solid arrows; induction of expression or activity=dashed arrow; inhibition of expression or activity=dashed blunt end; expression=arrowhead).

### DETAILED DESCRIPTION OF CERTAIN INVENTIVE EMBODIMENTS

Embodiments disclosed herein generally relate to diagnostic and prognostic methods for the detection of neurological conditions. Some methods relate to the discovery of biomarker ratios (for example, protein ratios) that are indicative of neurological conditions, such as Alzheimer's Disease (AD), mild AD, cognitive impairment, and brain microhemmorhages. Biomarkers include, for example, heme oxygenase 1 (HO1), biliverdin reductase (BLVR), estrogen receptor alpha (ERA), matrix metalloproteinase 9 (MMP9), superoxide dismutase (SOD), phosphorylated platelet derived growth factor receptor (Asp716), and S100A7. Accordingly, evaluating patient samples for the presence levels of such biomarkers can be an effective means of diagnosing neurological conditions and monitoring the progression of neurological conditions.

The terms “individual,” “host,” “subject” and “patient” are used interchangeably herein, and refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “ameliorating,” “treating,” “treatment,” “therapeutic,” or “therapy” do not necessarily mean total cure or abolition of the disease or condition. Any alleviation of any undesired signs or symptoms of a disease or condition, to any extent, can be considered amelioration, treatment and/or therapy. Furthermore, treatment may include acts that may worsen the patient's overall feeling of well-being or appearance.

The term “nucleic acids”, as used herein, may be DNA or RNA. Nucleic acids may also include modified nucleotides that permit correct read through by a polymerase and do not alter expression of a polypeptide encoded by that nucleic acid. The terms “nucleic acid” and “oligonucleotide” are used interchangeably to refer to a molecule comprising multiple nucleotides. As used herein, the terms refer to oligoribonucleotides as well as oligodeoxyribonucleotides. The terms shall also include polynucleosides (for example, a polynucleotide minus the phosphate) and any other organic base containing polymer. Nucleic acids include vectors, for example, plasmids, as well as oligonucleotides. Nucleic acid molecules can be obtained from existing nucleic acid sources, but are preferably synthetic (for example, produced by oligonucleotide synthesis).

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, for example, by the addition of carbohydrate residues to form glycoproteins. The terms “polypeptide,” “peptide” and “protein” include glycoproteins, as well as non-glycoproteins. Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (for example, 10 kDa) and/or when it cannot be produced by recombinant techniques (for example, not encoded by a nucleic acid sequence) and therefore involves different chemistry. Solid phase polypeptide synthesis procedures are well known in the art and further described by John Morrow Stewart and Janis Dillaha Young, Solid Phase Peptide Syntheses (2nd Ed., Pierce Chemical Company, 1984). Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing. In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

As used herein, a result is considered “significant” if the p value for the result is less than 0.05. In certain preferred embodiments, significant results have a p value less than 0.01, and even more preferably less than 0.001.

### Detection Methods

Some embodiments disclosed herein relate to diagnostic and prognostic methods for the detection of a neurological condition and/or monitoring the progression of a neurological condition. As used herein the phrase “diagnostic” means identifying the presence of or nature of a neurological condition. The detection of the level of expression of one or more biomarkers (for example, a first biomarker and a second biomarker) and the determination of a ratio of biomarkers (for example, the ratio of the first biomarker to the second biomarker) provides a means of diagnosing the neurological condition. Such detection methods may be used, for example, for early diagnosis of the condition, to determine whether a subject is predisposed to a neurological condition, to monitor the progress of the condition or the progress of treatment protocols, to assess the severity of the neurological condition, to forecast the an outcome of a neurological conditions and/or prospects of recovery, or to aid in the determination of a suitable treatment for a subject. The detection can occur in vitro, in situ, in silky, or in vivo.

The term “detect” or “measure” refers to identifying the presence, absence, amount, or level of the object to be detected (for example, a biomarker). As used herein, the term “level” refers to expression levels of RNA and/or protein or to DNA copy number of a biomarker. Typically, the level of the marker in a biological sample obtained from the subject is different (for example, increased or decreased) from a predetermined level (for example, the level of the same variant in a similar sample obtained from a healthy individual.

As used herein, “predetermined level” refers to the level of expression of a biomarker or to a ratio of biomarkers in a control sample (for example, a biological sample from a subject without a neurological condition). In some embodiments, the neurological condition can be diagnosed by assessing whether the biomarker expression or ratio of biomarkers varies from a predetermined level. For instance, the difference may be greater than, less than, equal to, or any number in between about 5%, 10%, 15%, 20%, 25%, 30%, 35%, 40%, 45%, 50%, 55%, 60%, 65%, 70%, 75%, 80%, 85%, 90%, 95%, 100%, 110%, 125%, 150%, 175%, 200%, 250%, 300%, 350%, 400%, 450%, 500%, 550%, 600%, 650%, 700%, 750%, 800%, 850%, 900%, 950%, 1,000%, 5,000%, 10,000%, 100,000% or greater. The predetermined level can be determined from a control. A control can be a sample or its equivalent from a normal patient or from a patient in a known disease state. For instance, the control can be from a patient with AD, MCI or brain microhemorrhages. The control can also be a standard or known amount of a reference biomarker (for example, protein or mRNA) or a standard or known amount of a ratio of biomarkers.

The term “about” or “approximately” means within an acceptable error range for the particular value as determined by one of ordinary skill in the art, which will depend in part on how the value is measured or determined, for example, the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

In some embodiments, labels can be used to aid in detection. For example, moieties (for example, antibodies) used to detect a biomarker can be labeled. The term “label” includes any moiety or item detectable by spectroscopic, photo chemical, biochemical, immunochemical, or chemical means. For example, useful labels include fluorescent dyes, radionuclides, phosphors, electron-dense reagents, enzymes, enzyme products (for example, chromagens catalytically processed by horseradish peroxidase or alkaline phosphatase commonly used in an ELISA or immunocytochemistry), biotin-avidin and streptavadin/polymer systems, dioxigenin, colloidal dye substances, fluorochromes, reducing substances, latexes, metals, particulates, dansyl lysine, antibodies, protein A, protein G, chromophores, haptens, and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, for example, incorporation of radioactive nucleotides, or biotinylated nucleotides that are recognized by avidin/streptavadin. The label may be directly or indirectly detectable. Indirect detection can involve the binding of a second label to the first label, directly or indirectly. For example, the label can be the ligand of a binding partner, such as biotin, which is a binding partner for avidin/streptavadin, or a nucleotide sequence, which is the binding partner for a complementary sequence, to which it can specifically hybridize. The binding partner may itself be directly detectable, for example, an antibody may be itself labeled with fluorescent molecules and/or enzymes (for example, HRP or alkaline phosphatase). The binding partner also may be indirectly detectable, for example, a nucleic acid having a complementary nucleotide sequence can be a part of a branched DNA molecule that is in turn detectable through hybridization with other labeled nucleic acid molecules (see, for example, P. D. Fahrlander and A. Klausner, Bio/Technology 6:1165 (1988)). Quantitation of the signal is achieved by, for example, scintillation counting, densitometry, flow cytometry and/or microscopical analysis with computer-algorithm assisted software(s).

Examples of detectable labels, optionally and preferably for use with immunoassays, include but are not limited to magnetic beads, fluorescent dyes, radiolabels, enzymes, chromagens catalytically processed by enzymes (for example, horseradish peroxide (HRP), alkaline phosphatase and others commonly used in an ELISA and immunocytochemisry), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Visualization of enzymes, (for example, HRP or alkaline phosphatase), can be achieved by means of using the enzymatic activity of the enzyme, for example, the oxidative-catalytic enzymatic activity of HRP or Alkaline phosphatase, to process and precipitate a substrate-chromogen. The final reaction product may be soluble in buffer or ethanol and may require stabilization to prevent fading. Chromogens that can be used include, but are not limited to 3,3′-diaminobenzidine tetrahydrochloride (DAB), Betazoid DAB, Cardassian DAB, 3,3′,5,5′-tetramethylbenzidine (TMB), benzidine dihydrochloride (BDHC) and p-phenylenediamine dihydrochloride with pyrocatechol (PPD-PC), 4-chloro-1-naphthol (4C1N), 3-amino-9-ethylcarbazole (AEC) and o-phenylenediamine (OPD), DAB-NI (Vector Laboratories), VECTOR® VIP (Vector Laboratories), VECTOR® SG (Vector Laboratories), VECTOR® RED (Vector Laboratories), VECTOR® BLACK (Vector Laboratories), VECTOR® BLUE (Vector Laboratories), BCIP/NBT (Vector Laboratories), Glucose oxidase NBT (Vector Laboratories), Glucose oxidase TNBT (Vector Laboratories), and Glucose oxidase INT (Vector Laboratories), Bajoran Purple, Romulin AEC, Ferangi Blue and Vulcan Fast Red (Biocare Medical Inc.). Some chromogens (for example, Bajoran Purple and VECTOR® RED) may also be used in double and triple stain procedures, nitrocellulose blots, and can be viewed by both bright- and darkfield microscopy. The visualization of the reaction product can be further improved by intensification with metal salts. At the light microscopic level, this intensification can enable color differentiation between distinct markers (see, for example, van der Want et al. Tract-tracing in the nervous system of vertebrates using horseradish peroxidase and its conjugates: tracers, chromogens and stabilization for light and electron microscopy. Brain Res Brain Res Protoc. 1997 August 1(3):269-79, which is hereby incorporated by reference in its entirety). In addition, the amounts of these precipitates can be semi-automatically or automatically quantified by algorithm based software (for example, Aperio Technology Inc, Vista, Calif.). Visualization can be achieved by using combinations of detectable labels in embodiments disclosed herein. For example, HRP can be used with alkaline phosphatase and visualized by microscopy (for example, bright—or dark-field microscopy) to differentiate between two or more distinct markers.

Examples of fluorescent dyes include, but are not limited to, 7-Amino-actinomycin D, Acridine orange, Acridine yellow, Alexa Fluor dyes (Molecular Probes), Auramine O, Auramine-rhodamine stain, Benzanthrone, 9,10-Bis(phenylethynyl)anthracene, 5,12-Bis(phenylethynyl)naphthacene, CFDA-SE, CFSE, Calcein, Carboxyfluorescein, 1-Chloro-9,10-bis(phenylethynyl)anthracene, 2-Chloro-9,10-bis(phenylethynyl)anthracene, Coumarin, Cyanine, DAPI, Dark quencher, Dioc6, DyLight Fluor dyes (Thermo Fisher Scientific), Ethidium bromide, Fluorescein, Fura-2, Fura-2-acetoxymethyl ester, Green fluorescent protein and derivatives, Hilyte Fluor dyes (AnaSpec), Hoechst stain, Indian yellow, Luciferin, Perylene, Phycobilin, Phycoerythrin, Phycoerythrobilin, Propidium iodide, Pyranine, Rhodamine, RiboGreen, Rubrene, Ruthenium(II) tris(bathophenanthroline disulfonate), SYBR Green, Stilbene, Sulforhodamine 101, TSQ, Texas Red, Umbelliferone, and Yellow fluorescent protein.

Examples of phsosphors include, but are not limited to Phosphor, Anthracene, Barium fluoride, Bismuth germanate, Cadmium sulfide, Cadmium tungstate, Gadolinium oxysulfide, Lanthanum bromide, Polyvinyl toluene, Scheelite, Sodium iodide, Stilbene, Strontium aluminate, Yttrium aluminium garnet, Zinc selenide, Zinc sulfide

Examples of radionuclides include, but are not limited to, ³²P, ³³P, ⁴³K, ⁴⁷Sc, ⁵²Fe, ⁵²Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ⁴³K, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi. Antibodies can be radiolabeled, for example, by the Iodogen method according to established methods.

A label may be chemically coupled directly to an antibody (for example, without a linking group) through an amino group, a sulfhydryl group, a hydroxyl group, or a carboxyl group. In some embodiments, a label can be attached to an antibody via a linking group. The linking group can be any biocompatible linking group, where “biocompatible” indicates that the compound or group can be non-toxic and may be utilized in vitro or in vivo without causing injury, sickness, disease, or death. The label can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

The protein biomarkers (for example, BLVR, BLVRB, ERA, S100A7, HO1, MMP9, and PDGFR) can be detected using a variety of methods known in the art. Some embodiments disclosed herein relate to methods of detecting a biomarker that is immunological in nature. “Immunological” refers to the use of antibodies (for example, polyclonal or monoclonal antibodies) specific for a biomaker. The phrase “specific for a biomarker,” “specifically binds to a biomarker,” or “specifically detects a biomarker” refers to, for example, antibodies that recognize the biomarker while not substantially cross-reacting with control samples containing other proteins. Antibodies specific for a biomarker include, but are not limited to, commercially available antibodies (for example, antibodies commercially available that recognize BLVR, BLVRB, ERA, S100A7, HO1, MMP9, and PDGFR) and those antibodies that can be produced by methods disclosed herein and by methods known in the art. Antibodies specific for the biomarkers can be produced readily using well known methods in the art. (See J. Sambrook, E. F. Fritsch and T. Maniatis, Molecular Cloning, a Laboratory Manual, second edition, Cold Spring Harbor Laboratory Press, pp. 18.7-18.18, 1989) For example, the biomarkers can be prepared readily using an automated peptide synthesizer. Next, injection of an immunogen (for example, a biomarker), such as (peptide)ₙ-KLH (n=1-30) in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated antibodies can be detected by using peptide fragments of the original immunogen.

The term “antibody” includes immunoglobulin molecules and immunologically active determinants of immunoglobulin molecules, for example, molecules that contain an antigen binding site which specifically binds (for example, immunoreacts with) an antigen. Structurally, the simplest naturally occurring antibody (for example, IgG) comprises four polypeptide chains, two copies of a heavy (H) chain and two of a light (L) chain, all covalently linked by disulfide bonds. Specificity of binding in the large and diverse set of antibodies is found in the variable (V) determinant of the H and L chains; regions of the molecules that are primarily structural are constant (C) in this set. The term “antibody” but is not limited to, polyclonal antibodies, monoclonal antibodies, whole immunoglobulins, and antigen binding fragments of the immunoglobulin.

The binding sites of the proteins that comprise an antibody, for example, the antigen-binding functions of the antibody, are localized by analysis of fragments of a naturally-occurring antibody. Thus, antigen-binding fragments are also intended to be designated by the term “antibody.” Examples of binding fragments encompassed within the term antibody include: a Fab fragment consisting of the VL, VH, CL and CH1 domains; an F, fragment consisting of the VH and CH1 domains; an Fᵥ fragment consisting of the V_{L} and V_{H} domains of a single arm of an antibody; a dAb fragment (Ward et al., 1989 Nature 341:544-546) consisting of a VH domain; an isolated complementarity determining region; and an F(ab′)₂ fragment, a bivalent fragment comprising two Fab′ fragments linked by a disulfide bridge at the hinge region. These antibody fragments are obtained using conventional techniques well-known to those with skill in the art, and the fragments are screened for utility in the same manner as are intact antibodies. The term “antibody” is further intended to include bispecific and chimeric molecules having at least one antigen binding determinant derived from an antibody molecule, as well as single chain (scFv) antibodies. The term “single-chain Fv,” also abbreviated as “sFv” or “scFv,” refers to antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

Quantification assays for a biomarker and detection of a biomarker can use binding molecules specific for the biomarker other than antibodies, including but not limited to, affibodies, aptamers or other specific binding molecules known in the art.

Examples of acceptable immunoassays include, for example, ELISA, radioimmunoassay, immunofluorescent assay, “sandwich” immunoassay, western blot, immunoprecipitation assay and immunoelectrophoresis assays. In other aspects, microbeads, arrays, microarrays, etc. can be used in detecting the LMW peptides. Examples of acceptable assays include, but are not limited to, a suspension bead assay (Schwenk et al., “Determination of binding specificities in highly multiplexed bead-based assays for antibody proteomics,” Mol. Cell. Proteomics, 6(1): 125-132 (2007)), an antibody microarray (Borrebaeck et al., “High-throughput proteomics using antibody microarrays: an update,” Expert Rev. Mol. Diagn. 7(5): 673-686 (2007)), an aptamer array (Walter et al., “High-throughput protein arrays: prospects for molecular diagnostics,” Trends Mol. Med. 8(6): 250-253 (2002)), an affybody array (Renberg et al., “Affibody molecules in protein capture microarrays: evaluation of multidomain ligands and different detection formats,” J. Proteome Res. 6(1): 171-179 (2007)), and a reverse phase array (VanMeter et al., “Reverse-phase protein microarrays: application to biomarker discovery and translational medicine,” Expert Rev. Mol. Diagn. 7(5): 625-633 (2007)). All of these publications are incorporated herein by reference.

In other embodiments, the biomarkers can be detected using mass spectrometry (MS). One example of this approach is tandem mass spectrometry (MS/MS), which involves multiple steps of mass selection or analysis, usually separated by some form of fragmentation. Most such assays use electrospray ionization followed by two stages of mass selection: a first stage (MS1) selecting the mass of the intact analyte (parent ion) and, after fragmentation of the parent by collision with gas atoms, a second stage (MS2) selecting a specific fragment of the parent, collectively generating a selected reaction monitoring assay. In one embodiment, collision-induced dissociation is used to generate a set of fragments from a specific peptide ion. The fragmentation process primarily gives rise to cleavage products that break along peptide bonds. Because of the simplicity in fragmentation, the observed fragment masses can be compared to a database of predicted masses for known peptide sequences. A number of different algorithmic approaches have been described to identify peptides and proteins from tandem mass spectrometry (MS/MS) data, including peptide fragment fingerprinting (SEQUEST, MASCOT, OMSSA and X!Tandem), peptide de novo sequencing (PEAKS, LuteFisk and Sherenga) and sequence tag based searching (SPIDER, GutenTAG).

In some embodiments, multiple reaction monitoring (MRM) can be used to identify the biomarkers in patient samples. This technique applies the MS/MS approach to, for example, tryptic digests of the input sample, followed by selected ion partitioning and sampling using MS to make the analyte selection more objective and discrete by following the exact m/z ion of the tryptic fragment that represents the analyte. Such an approach can be performed in multiplex so that multiple ions can be measured at once, providing an antibody-free method for analyte measurement. See, for example, Andersen et al., Molecular & Cellular Proteomics, 5.4: 573-588 (2006); Whiteaker et al., J. Proteome Res. 6(10): 3962-75 (2007). Both publications are incorporated herein by reference.

In further embodiments, the biomarkers can be detected using nanoflow reverse-phase liquid chromatography-tandem mass spectrometry. See, for example, Domon B, Aebersold R. Science, 312(5770:212-7 (2006), which is incorporated herein by reference. Using this approach, practitioners obtain peptide fragments, usually by trypsin digest, and generate mass spectrograms of the fragments, which are then compared to a database, such as SEQUEST, for protein identification.

In other aspects, the biomarkers can be detected using immuno-mass spectrometry. See, for example, Liotta L et al. J Clin Invest., 116(1):26-30 (2006) and Nedelkov, Expert Rev. Proteomics, 3(6): 631-640 (2006), which are incorporated herein by reference. Immuno-mass spectrometry provides a means for rapidly determining the exact size and identity of a peptide biomarker isoform present within a patient sample. When developed as a high throughput diagnostic assay, a drop of patient's blood, serum or plasma can be applied to a high density matrix of microcolumns or microwells filled with a composite substratum containing immobilized polyclonal antibodies, directed against the peptide marker. All isoforms of the peptide that contain the epitope are captured. The captured population of analytes including the analyte fragments are eluted and analyzed directly by a mass spectrometer such as MALDI-TOF MS. The presence of the specific peptide biomarker at its exact mass/charge (m/z) location can be used as a diagnostic test result. The analysis can be performed rapidly by simple software that determines if a series of ion peaks are present at defined m/z locations.

In yet more embodiments, the biomarkers can be detected using standard immunoassay-based approaches whereby fragment specific antibodies are used to measure and record the presence of the diagnostic fragments. See, for example, Naya et al. “Evaluation of precursor prostate-specific antigen isoform ratios in the detection of prostate cancer.” Urol Oncol. 23(1):16-21 (2005). Moreover, additional immunoassays are well known to one skilled in the field, such as ELISA (Maeda et al., “Blood tests for asbestos-related mesothelioma,” Oncology 71: 26-31 (2006)), microfluidic ELISA (Lee et al., “Microfluidic enzyme-linked immunosorbent assay technology,” Adv. Clin. Chem. 42: 255-259 (2006)), nanocantilever immunoassay (Kurosawa et al., “Quartz crystal microbalance immunosensors for environmental monitoring,” Biosens Bioelectron, 22(4): 473-481 (2006)), and plasmon resonance immunoassay (Nedelkov, “Development of surface Plasmon resonance mass spectrometry array platform,” Anal. Chem. 79(15): 5987-5990 (2007)). All publications are incorporated herein by reference.

In further embodiments, the biomarkers can be detected using electrochemical approaches. See, for example, Lin et al., Anal. Sci. 23(9): 1059-1063 (2007)), which is hereby incorporated by reference in its entirety.

In some embodiments, the expression of a biomarker can be detected by measuring levels of mRNA encoding a protein biomarker. Any technique known in the art can be used to detect mRNA levels of biomarkers. Those of skill in the art are well acquainted with methods of mRNA detection, for example, via the use of complementary hybridizing primers (for example, labeled with radioactivity or fluorescent dyes) with or without polymerase chain reaction (PCR) amplification of the detected products, followed by visualization of the detected mRNA via, for example, electrophoresis (for example, gel or capillary); by mass spectroscopy; etc. The level of mRNA may also be measured, for example, using ethidium bromide staining of a standard RNA gel, Northern blotting, primer extension, or a nuclease protection assay. Other means of detecting the expression profile of mRNA encoding a protein biomarker include, but are not limited to, PCR-based methods (for example, quantitative real time PCR), microarray based methods, and ribonuclease protection assays (RPA).

Additional means of detecting the expression of a biomarker include, but are not limited to, detecting the level of promoter modification (for example, methylation) and detecting the level of histone modification. For example, promoter methylation has been shown to correlate with mRNA expression (see, for example, Lindsey et al. 2007 Jul. 16; 97(2):267-74).

Further means of detecting the expression of a biomarker include, but are not limited to, determining the level DNA encoding the biomarker. These methods include, but are not limited to, various approaches for DNA sequencing (to find, for example mutations or deletions) and other approaches known in the art.

### Ratios of Biomarkers

In some embodiments, one ratio of biomarkers can be determined and used for diagnosis. In other embodiments, more than one ratio of biomarkers can be evaluated simultaneously. For example, ratios of ERA/BLVR, MMP9/BLVR, BLVRB/BLVR, HO1/BLVR, PDGFR/BLVR, S100A7/BLVR, ERA/BLVRB, HO1/BLVRB, MMP9/HO1, PDGFR/HO1, and/or S100A7/ERA can be evaluated individually or in any combination. In additional embodiments, the ratios of biomarkers can be used in combination with one or more of the biomarkers disclosed in international Application No. PCT/US2007/023026, published as WO 2008/063369, which is hereby incorporated by reference in its entirety. For example, greater than or equal to or any number in between about 2, 5, 10, 20, 30, 50, 75, 100, 500 biomarkers and/or ratios of biomarkers can be evaluated according to the methods described herein. In some embodiments, analyzing more than one biomarker or ratio of biomarkers can increase accuracy of the diagnosis.

The methods described herein can be combined with any known diagnostic techniques to increase the accuracy of the diagnosis. For example, some of the methods described herein can be combined with neuroimaging techniques for the detection of neuropathy and brain microvasculopathy associated with a neurological condition. In some embodiments, neuroimaging can be used to detect brain microhemorrages associated with cognitive impairment. Using magnetic resonance imaging, focal signal intensity losses secondary to iron-containing hemosiderin residuals can be detected. These spots on the MR image have been termed “signal voids,” “susceptibility artifacts,” “black holes,” “dots,” “microbleeds,” “old microbleeds” (OMBs), “multifocal signal loss lesions” or “microhemorrhages” (MH). Generically, these spots are called small hypointensities (SH) and are associated with AD and MCI (Cordonnier et al. Neurology (2006) 66:1356-1360; Werring et al. Brain (2004) 127:2265-2275). Suitable MR imaging techniques include gradient refocused echo T₂* (GRE-T₂) and susceptibility weighted imaging (SWI).

Neuroimaging methods that detect metabolic changes in the brain also can be used in conjunction with the biomarkers described herein. MR spectroscopy that detects, for example, differences in neurotransmitters, such as glutamine, glutamate and gamma-aminobutryic acid (GABA), can be used to analyze changes in these systems associated with a neurological condition. These metabolic changes can be correlated with cognitive decline and/or biomarker levels.

### Monitoring Neurological Conditions

Some embodiments disclosed herein relate to methods for monitoring the progress of a neurological condition. For example, levels of one or more ratios of biomarkers can be determined in a biological sample of a subject at two or more distinct times. The ratios of biomarkers can be compared to determine the progress of the neurological condition. In some embodiments, the efficacy of a treatment for a neurological condition in a subject is determined. For example, the level of a ratio of biomarkers in subjects or biological sample from the subject is determined before a treatment for the neurological condition and compared to the level of the ratio of biomarkers in the subject or biological sample of the subject during or after the treatment for the neurological condition. In this way, it is possible to evaluate the effectiveness of the therapy and determine future treatments.

Any information disclosed herein (for example, data from assays, such as the expression level of a biomarker or the determination of one or more ratios of biomarkers) can be stored, recorded, and manipulated on any medium that can be read and accessed by a computer. As used herein, the words “recorded” and “stored” refer to a process for storing information on computer readable medium. A skilled artisan can readily adopt any of the presently known methods for recording information on a computer readable medium to generate manufactures comprising the information of this embodiment. A variety of data storage structures are available to a skilled artisan for creating a computer readable medium having recorded thereon information (for example, data from assays, such as the expression level of a biomarker or one or more ratios of biomarkers). The choice of the data storage structure will generally be based on the component chosen to access the stored information. Computer readable media include magnetically readable media, optically readable media, or electronically readable media. For example, the computer readable media can be a hard disc, a floppy disc, a magnetic tape, zip disk, CD-ROM, DVD-ROM, RAM, or ROM as well as other types of other media known to those skilled in the art. The computer readable media on which the sequence information is stored can be in a personal computer, a network, a server or other computer systems known to those skilled in the art.

Some embodiments utilize computer-based systems that contain the information described herein and convert this information into other types of usable information (for example, models for diagnosis, prognosis, or determining suitable treatments). The term “a computer-based system” refers to the hardware, software, and any database used to analyze information (for example, data from assays, such as the expression level of a biomarker or one or more ratios of biomarkers). The computer-based system preferably includes the storage media described above, and a processor for accessing and manipulating the data. The hardware of the computer-based systems of this embodiment comprises a central processing unit (CPU) and a database. A skilled artisan can readily appreciate that any one of the currently available computer-based systems are suitable.

In some embodiments, the computer system includes a processor connected to a bus that is connected to a main memory (preferably implemented as RAM) and a variety of secondary storage devices, such as a hard drive and removable medium storage device. The removable medium storage device can represent, for example, a floppy disk drive, a DVD drive, an optical disk drive, a compact disk drive, a magnetic tape drive, etc. A removable storage medium, such as a floppy disk, a compact disk, a magnetic tape, etc. containing control logic and/or data recorded therein can be inserted into the removable storage device. The computer system includes appropriate software for reading the control logic and/or the data from the removable medium storage device once inserted in the removable medium storage device. Information described herein can be stored in a well known manner in the main memory, any of the secondary storage devices, and/or a removable storage medium. Software for accessing and processing this information (such as search tools, compare tools, and modeling tools etc.) reside in main memory during execution.

As used herein, “a database” refers to memory that can store any information described herein (for example, levels of biomarker expression, ratios of biomarkers, and values, levels, or results from assays). Additionally, a “database” refers to a memory access component that can access manufactures having recorded thereon information described herein. In other embodiments, a database stores a “biomarker expression profile” comprising the values, levels, ratios and/or results from one or more assays or methods, as described herein or known in the art, and relationships between these values, levels, ratios, and/or results. The data and values or results from assays can be stored and manipulated in a variety of data processor programs in a variety of formats. For example, the sequence data can be stored as text in a word processing file, an html file, or a pdf file in a variety of database programs familiar to those of skill in the art.

A “search program” refers to one or more programs that are implemented on the computer-based system to compare information (for example, levels of biomarker expression or one or more ratios of biomarkers). A search program also refers to one or more programs that compare one or more pieces of information (for example, levels of biomarker expression or ratios of biomarkers) to other information that exist in a database. A search program is used, for example, to compare levels of biomarker expression or ratios of biomarkers to predetermined levels that are present in one or more databases. Still further, a search program can be used to compare values, levels or results from assays described herein.

A “retrieval program” refers to one or more programs that can be implemented on the computer-based system to obtain a profile of biomarker expression. Further, a profile can have one or more symbols that represent these biomarkers including, but not limited to values, levels, or results from an assay.

### Neurological Conditions

The neurological condition or disease being detected according to the methods described herein can be, for example, Alzheimer's disease (AD), mild cognitive impairment (MCI), stable mild cognitive impairment (stable MCI), mild AD, vascular dementia (VD), angiopathy black holes, cerebral amyloid angiopathy (CAA) and brain microhemorrhages. Unless otherwise indicated, the conditions and activities noted herein refer to the commonly accepted definitions thereof. For instance, as described in more detail in the Examples, cognitive impairment is defined according to the Mayo Clinic criteria.

Levels of biomarkers and/or ratios of biomarkers described herein can be useful in detecting a neurological condition during its early stages, such as while the condition is still associated with MCI or mild AD or for detecting brain vasculopathy, such as brain microhemorrhages. Conditions can be classified according to various criteria and/or cognitive tests known in the art (See, for example, Petersen R C J Intern Med (2004) 256:183-194; Petersen et al. (1999) Arch Neurol 56:303-308; Reisberg B (2007) Int Psychogeriatr 19:421-456). Cognitive tests include, for example, Logical Memory I and II, Wisconsin Card Sorting Test, Trail Making Test A and B, Boston Naming Test, Draw-A Clock, Geriatric Depression Scale, Word Fluency (Phonemic and Semantic) and videotaped Global Clinical Dementia Rating (CDR) with informant. Mild cognitive impairment (MCI) cases can fulfill the Mayo Clinic criteria for classification as MCI-multiple domain impairment (MCI-MCDI) with the following characteristics: i) A memory complaint confirmed by either corrected Logical Memory testing or reports of the informant and a Clinical Dementia Rating (CDR)=0.5. ii) Normal activities of daily living. iii) Normal general cognitive function. iv) Abnormal memory for age as measured by standard scores and education. v) A global CDR of 0.5 and no dementia. vi) No history of significant vascular problems, insulin-requiring diabetes, or uncontrolled hypertension. Meanwhile, stable mild cognitive impairment (stable MCI) can be classified based on a sum of boxes=0.5−3.5 on several evaluations, CDR logical memory impairment with logical memory impairment on at least one evaluation, and/or neuropsychological testing in MCI range inconsistently and clinical judgment. Progression to dementia (mild AD) can be classified by a sum of CDR boxes of 3.5 or more, NINCDS-ARDRDA criteria, neuropsychological tests congruent with CDR, a Logical Memory raw score low to zero and/or clinical judgment. The parameters described above can be useful in identifying subjects at risk of a neurological condition.

In other embodiments, the biomarker can be a peptide associated with a metabolic pathway or cellular process. In further embodiments, the biomarker is a peptide associated with inflammation, estrogen activity, pigment epithelium-derived factor (PEDF) vitamin D metabolism and bone mineralization, coagulation and platelet activity, the complement cascade, acyl-peptide hydrolase (APH) activity, vitamin A and thyroxine, phospholipase activity, globin activity, glycosylation or is glycosylated, protease inhibition, keratins and related proteins, heme degradation, pyruvate metabolism, calcium related proteins, defensin, gelsolin, vitronectin, profilin, thrombospondin, peroxiredoxin, alcohol dehydrogenase, apolipoproteins, iron and copper metabolism, or NMDA receptor-related proteins.

In some embodiments, the biomarkers, ratios of biomarkers, and antibodies described herein are useful for discovering novel aspects of neurological conditions, such as those described herein.

### Biological Samples

In some embodiments, the biomarkers are harvested from a biological sample prior to their detection. Numerous well known tissue or fluid collection methods can be utilized to collect the biological sample from the subject in order to determine the level of DNA, RNA and/or protein or fragment thereof of the biomarker(s) of interest in the subject and to determine ratios of particular biomarkers. Biological samples can include, for example, blood, serum, plasma, urine, lymph, tissue and products thereof.

For example, the protein biomarkers can be harvested from a sample using a capture-particle that comprises a molecular sieve portion and an analyte binding portion. Briefly, either the molecular sieve portion or the analyte binding portion or both comprise a cross-linked region having modified porosity, or pore dimensions sufficient to exclude high molecular weight molecules. Examples of such suitable methods are described, for example, in PCT Pub. No. WO/2008/115653, filed Feb. 21, 2008 and PCT Pub. No. WO/2007/038523, filed Sep. 27, 2006, both of which are incorporated herein by reference.

In another embodiment, the protein biomarkers are digested prior to detection, so as to reduce the size of the peptides. Such digestion can be carried out using standard methods well known in the field. Examples of acceptable treatments include, but are not limited to, enzymatic and chemical treatments. Such treatments can yield partial as well as complete digestions. One example of an enzymatic treatment is a trypsin digestion.

Additional methods for obtaining a biological sample include, but are not limited to, fine needle biopsy, needle biopsy, core needle biopsy and surgical biopsy (for example, brain biopsy), lavage, and any known method in the art. Regardless of the procedure employed, once a biopsy/sample is obtained, biomarker(s) may be identified, the level of the biomarker(s) can be determined, one or more ratios can be calculated, and one or more neurological conditions may be identified and/or monitored and/or treated.

### Kits

Some embodiments disclosed herein provide for a kit for use in, for example, the screening, diagnosis, or monitoring the progress of a neurological condition. Such a kit may comprise a first agent or binding moiety (for example, an antibody, such as a primary antibody) which specifically detects or binds to a first biomarker (for example, BLVR, BLVRB, ERA, S100A7, HO1, MMP9, or PDGFR), a second agent or binding moiety (for example, an antibody, such as a primary antibody) which specifically detects or binds to a first biomarker (for example, BLVR, BLVRB, ERA, S100A7, HO1, MMP9. or PDGFR), and instructions for use. Such a kit may further comprise a reaction container, various buffers, additional agents or binding moieties, and the like. In some embodiments, the first agent or binding moiety is labeled. In some embodiments, the second agent or binding moiety is labeled. In one embodiment, the kit further comprises additional agents or binding moieties (for example, secondary antibodies) which binds specifically to the first binding moiety and/or second binding moiety.

In some embodiments, the kit may comprise a reference sample, for example, a negative and/or positive control. In that embodiment, the negative control would be indicative of the absence of the neurological condition and the positive control would be indicative of the neurological condition. A large number of control samples can be assayed to establish the threshold, mode and width of the distribution of a biomarker or one or more ratios of biomarkers in a normal biological sample against which test biological samples are compared. These data can be provided to users of the kit.

In one embodiment, the agents or the binding moieties in the kit can be antibodies or fragments thereof which specifically bind to the biomarkers. In these kits, antibodies (for example, primary and/or secondary antibodies) may be provided with means for binding to detectable marker moieties (for example, labels) or substrate surfaces. Alternatively, the kits may include antibodies already bound to marker moieties (for example, labels) or substrates. Antibodies and binding fragments thereof can be, for example, lyophilized or in solution. Additionally, the preparations can contain stabilizers to increase the shelf-life of the kits, for example, bovine serum albumin (BSA). Wherein the antibodies and antigen binding fragments thereof are lyophilized, the kit can contain further preparations of solutions to reconstitute the preparations. Acceptable solutions are well known in the art, for example, PBS. In some embodiments, the antibody is a polyclonal antibody, a monoclonal antibody, a humanized antibody, a chimeric antibody, a recombinant antibody, or fragment thereof.

In some embodiments, the kits can further include the components for an immunohistochemical assay for measuring the biomarker and/or fragments thereof. For example, kits containing antibody bound to multiwell microtiter plates can be provided. The kit may include a standard or multiple standard solutions containing a known concentration of biomarker or other proteins for calibration of the assays. Samples to be tested in this application include, for example, blood, serum, plasma, urine, lymph, tissue and products thereof.

Alternatively, the kits can be used in immunoassays, such as immunohistochemistry to test subject tissue biopsy sections. The kits may also be used to detect the presence of one or more biomarkers in a biological sample obtained from a subject using immunohistocytochemistry.

The compositions of the kit can be formulated in single or multiple units for either a single test or multiple tests. The kits can be used to determine one or more ratios of biomarkers.

The above-mentioned kit can be used for the detection of any neurological condition including, without limitation, Alzheimer's disease, mild cognitive impairment, stable mild cognitive impairment, mild Alzheimer's disease, vascular dementia, angiopathy black holes, cerebral amyloid angiopathy, and microhemorrages. The kit may also be used to determine the severity, aggressiveness or grade of the neurological condition. In some embodiments, a kit may also be used for identifying potential candidate therapeutic agents for treating the neurological condition.

Each reference disclosed herein, and throughout the specification, is incorporated by reference in its entirety. The following examples provide illustrations of some of the embodiments described herein but are not intended to limit the disclosure.

The Examples below describe in further detail the identification of proteins and protein ratios that can be used as potential biomarkers for neurological conditions.

### Example 1

Alzheimer's disease (AD) is the most common form of dementia with an estimated 18 million affected patients (Mount et al. Nat. Med. (2006) 12:780-784). Although this neurodegenerative disease was identified over 100 years ago, the molecular principles behind AD are not fully understood. Furthermore, the only method of a certain diagnosis of AD is postmortem brain histochemical staining for clinical hallmarks such as neurofibrillary tangles and β-amyloid deposits in the parenchyma and blood vessel walls. Although several therapies for AD are being tested in clinical trials, there is no biomarker available to estimate the effectiveness of treatment. Moreover, AD must be diagnosed early to therapeutically prevent neurodegeneration, which is largely irreversible. Mild cognitive impairment (MCI) has been recognized as a transitional stage between normal aging and AD (Petersen R C J Intern Med (2006) 256:183-194) and may offer the critical treatment window before significant and irreversible neurodegeneration takes place. Thus, a biomarker for MCI could provide significant clinical utility for individualizing therapy.

Much effort has been done to establish clinical biomarkers for AD based on imaging, for example, with in vivo magnetic resonance (MR) imaging. For example, hippocampal atrophy is used to aid in the diagnosis of AD as well as the prediction of which MCI patients will eventually progress into AD (Schott et al. Curr. Opin. Neurol. (2006) 19:552-558). However, a major drawback is the significant fluctuation between individuals, which makes sequential measurements over a period of time necessary for a correct interpretation of results. Another source of biomarkers that has been studied extensively in AD is cerebrospinal fluid (CSF) (Castaño et al. Neurol. Res. (2006) 28:155-63). However, specificity and sensitivity vary between studies and the correct differentiation between different types of dementia has been difficult to ascertain (Nagy Z Biochim Biophys Acta (2007) 1772:402-408).

Peripheral blood, serum, or plasma offer several advantages as potential biomarker sources. It is much more accessible and therefore could easily be tested in a regular clinical setting. Furthermore, during the initial biomarker discovery phase, serum or plasma can be collected from patients at different stages of the disease, whereas ante-mortem CSF samples are much harder to obtain. Moreover, multiple alterations have already been observed in AD blood, such as altered gene expression profiles in AD lymphocytes (Palotas et al. Brain Res. Bull. (2002) 58:203-205) (Kalman et al. Psychiatr. Genet. (2005) 15:1-6), increased serum copper (Squitti et al. Neurology (2005) 64:1040-1046), as well as increased membrane fluidity and an abnormal expression pattern of APP isoforms in AD platelets (Kozubski et al. Alzheimer Dis. Assoc. Disord. (2002) 16:52-54).

Studies applying a serum proteome screening approach are not limited by the current incomplete understanding of the mechanisms involved in AD. Although most of the protein mass in serum consists of a few highly abundant proteins such as albumin and immunoglobulins, it is the low abundant, low molecular weight (LMW) proteome, which consists of cleavage fragments and proteins small enough to enter the blood stream passively, that may contain the biomarkers that could identify a disease (Liotta et al. Nature (2003) 425:905). Whereas some studies have approached this issue by using two dimensional gel electrophoresis coupled with mass spectrometry (MS) (Hye et al. Brain (2006) 129:3042-3050), a method that focuses on the detection of LMW proteins and protein fragments complexed with high abundant serum proteins has been previously developed (Lowenthal et al. Clin. Chem. (2005) 51:1933-1945). A similar technique has been independently applied by Lopez et al. to successfully identify unique mass fingerprints in AD serum (Lopez et al. Clin. Chem. (2005) 51:1946-1954). Combining “free” and complexed LMW proteins and protein fragments, and using liquid chromatography coupled tandem mass spectrometry (LC-MS/MS), studies described herein identify proteins and peptides that are unique to AD, MCI or normal serum. To achieve this, serum obtained from a community based cohort of cognitively normal, MCI and mild AD subjects was used.

### Mass Spectrometry

Liquid chromatography coupled tandem mass spectrometry LC-MS/MS was used to identify candidate protein AD biomarkers in unfractionated serum and LMW serum protein fractions from cognitively normal, MCI and mild AD subjects, as well as in LMW serum protein fractions of three subjects before and after cognitive decline. The differential abundances of selected candidate protein biomarkers were verified using RPPAs.

### Unfractionated Serum

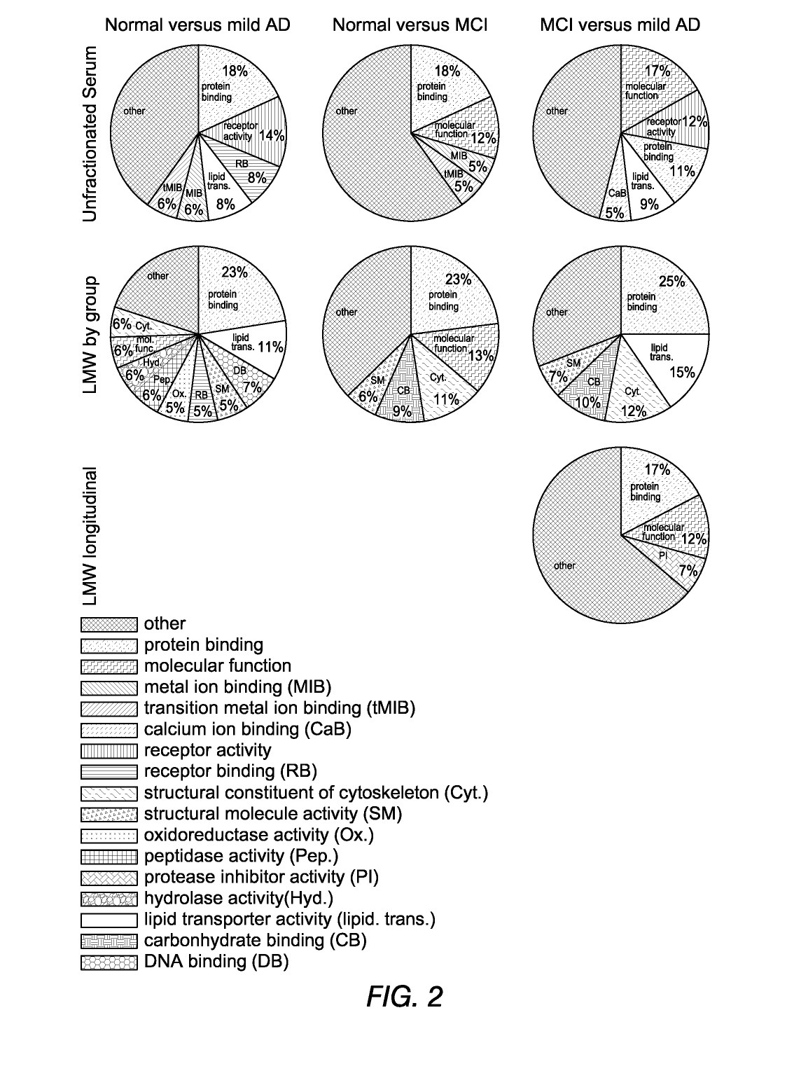

Unfractionated serum from 7 cognitively normal, 5 MCI and 12 mild AD participants was subjected to LC-MS/MS analysis. Combining all identified proteins in functional protein classes (GO terms), no prominent differences in protein categories between Normal, MCI and mild AD sera were found (filter criteria applied to the database search results: detection of 4 spectra per protein, present in at least 66% of samples per category, with a probability score <1.00E-03). The majority of proteins in serum were not expected to change based on a disease originating in the brain. After selection of peptides that differed between Normal, MCI and mild AD sera, several functional categories differentially represented between disease groups were found (FIG. 2; Tables 1-3). Three of these categories were: “metal/transition metal binding” proteins, proteins with “lipid transporter activity” and proteins with associated “receptor activity or receptor binding”. All three categories were represented in the group of peptides with different abundances in Normal versus mild AD sera. Metal/transition metal binding proteins were found to be differentially abundant in sera from Normal and MCI patients, whereas lipid transporter activity proteins, receptor activity proteins, and calcium binding proteins were differentially abundant in MCI and mild AD patient sera samples.

TABLE 1
Unfractionated serum proteins that were identified in a significantly
higher or lower number of mild AD participants than Normals.
(Normal (n = 7), MCI (n = 5) and mild AD (n = 12))

| | | | peptides identified in | | | % difference |
|---|---|---|---|---|---|---|
| Regulation | Accession | Protein | Normal | MCI | mild AD | mild AD/Normal |
| ↑ | P16591 | Proto-oncogene tyrosine-protein kinase FER | 0% | 40% | 67% | 200% |
| ↑ | P22792 | Carboxypeptidase N subunit 2 precursor | 0% | 60% | 58% | 200% |
| ↑ | Q6ZVQ3 | Hypothetical protein FLJ42220 | 0% | 80% | 50% | 200% |
| ↑ | Q9H212 | HNRBF-2 | 0% | 20% | 33% | 200% |
| ↑ | P13667 | Protein disulfide-isomerase A4 precursor | 0% | 20% | 33% | 200% |
| ↑ | O60602 | Toll-like receptor 5 precursor | 0% | 0% | 33% | 200% |
| ↑ | O95793 | Double-stranded RNA-binding protein Staufen | 0% | 40% | 33% | 200% |
| | | homolog | | | | |
| ↑ | Q7Z3Z2 | Protein C1 orf36 | 0% | 20% | 33% | 200% |
| ↑ | Q05513 | Protein kinase C, zeta type | 0% | 40% | 33% | 200% |
| ↑ | Q4V312 | Colony stimulating factor 2 receptor, alpha, low- | 0% | 60% | 33% | 200% |
| | | affinity | | | | |
| ↑ | Q93088 | Betaine--homocysteine S-methyltransferase | 14% | 60% | 75% | 136% |
| ↑ | Q8N7W7 | Hypothetical protein FLJ40259 | 14% | 40% | 58% | 121% |
| ↑ | P35542 | Serum amyloid A-4 protein precursor | 14% | 0% | 58% | 121% |
| ↑ | Q9BXB9 | LIM mineralization protein 2 | 14% | 60% | 50% | 111% |
| ↓ | Q99996 | A-kinase anchor protein 9 | 43% | 0% | 0% | 200% |
| ↓ | P09758 | Tumor-associated calcium signal transducer 2 | 43% | 40% | 8% | 135% |
| | | precursor | | | | |
| ↓ | Q5VVM6 | Novel protein | 43% | 20% | 8% | 135% |
| ↓ | P12814 | Alpha-actinin 1 | 43% | 0% | 8% | 135% |

TABLE 2
Unfractionated serum proteins that were identified in a significantly
higher or lower number of mild AD than MCI participants.
(Normal (n = 7), MCI (n = 5) and mild AD (n = 12))

| | | | peptides identified in | | | % difference |
|---|---|---|---|---|---|---|
| Regulation | Accession | Protein | Normal | MCI | mild AD | mild AD/Normal |
| ↑ | P35542 | Serum amyloid A-4 protein precursor | 14% | 0% | 58% | 200% |
| ↑ | P02766 | Transthyretin precursor | 71% | 0% | 50% | 200% |
| ↑ | P09874 | Poly [ADP-ribose] polymerase 1 | 29% | 0% | 42% | 200% |
| ↑ | P01764 | Ig heavy chain V-III region VH26 precursor | 43% | 0% | 42% | 200% |
| ↑ | Q9NPP6 | Immunoglobulin heavy chain variant | 43% | 0% | 33% | 200% |
| ↑ | Q96MA6 | Hypothetical protein FLJ32704 | 29% | 0% | 33% | 200% |
| ↑ | Q9NYQ6 | Cadherin EGF LAG seven-pass G-type | 43% | 0% | 33% | 200% |
| | | receptor 1 precursor | | | | |
| ↑ | O60602 | Toll-like receptor 5 precursor | 0% | 0% | 33% | 200% |
| ↑ | Q15166 | Serum paraoxonase/lactonase 3 | 14% | 0% | 33% | 200% |
| ↓ | O60687 | Sushi-repeat-containing protein, X-linked 2 | 0% | 40% | 0% | 200% |
| ↓ | Q6N092 | Hypothetical protein DKFZp686K18196 | 14% | 40% | 0% | 200% |
| ↓ | Q96S24 | Hypothetical protein gs30 | 0% | 40% | 0% | 200% |
| ↓ | P20701 | Integrin alpha-L precursor | 0% | 60% | 8% | 151% |
| ↓ | Q8N549 | Hypothetical protein C8orf36 | 14% | 40% | 8% | 131% |
| ↓ | P09758 | Tumor-associated calcium signal transducer 2 | 43% | 40% | 8% | 131% |
| | | precursor | | | | |
| ↓ | P10643 | Complement component C7 precursor | 29% | 80% | 25% | 105% |

TABLE 3
Unfractionated serum proteins that were identified in a significantly higher or lower
number of MCI participants than Normals. (Normal (n = 7), MCI (n = 5) and
mild AD (n = 12))

| | | | peptides identified in | | | % difference |
|---|---|---|---|---|---|---|
| Regulation | Accession | Protein | Normal | MCI | mild AD | mild AD/Normal |
| ↑ | Q6ZVQ3 | Hypothetical protein FLJ42220 | 0% | 80% | 50% | 200% |
| ↑ | Q4V312 | Colony stimulating factor 2 receptor, alpha, low- | 0% | 60% | 33% | 200% |
| | | affinity | | | | |
| ↑ | P20701 | Integrin alpha-L precursor | 0% | 60% | 8% | 200% |
| ↑ | P22792 | Carboxypeptidase N subunit 2 precursor | 0% | 60% | 58% | 200% |
| ↑ | O95793 | Double-stranded RNA-binding protein Staufen | 0% | 40% | 33% | 200% |
| | | homolog | | | | |
| ↑ | P16591 | Proto-oncogene tyrosine-protein kinase FER | 0% | 40% | 67% | 200% |
| ↑ | Q05513 | Protein kinase C, zeta type | 0% | 40% | 33% | 200% |
| ↑ | O60687 | Sushi-repeat-containing protein, X-linked 2 | 0% | 40% | 0% | 200% |
| ↑ | Q7Z2W7 | Transient receptor potential cation channel | 0% | 40% | 25% | 200% |
| | | subfamily M | | | | |
| ↑ | Q96S24 | Hypothetical protein gs30 | 0% | 40% | 0% | 200% |
| ↑ | P36955 | Pigment epithelium-derived factor precursor | 14% | 80% | 33% | 139% |
| ↑ | P10720 | Platelet factor 4 variant precursor | 86% | 20% | 58% | 124% |
| ↑ | Q93088 | Betaine--homocysteine S-methyltransferase | 14% | 60% | 75% | 123% |
| ↑ | Q9H943 | Protein C10orf68 | 14% | 60% | 33% | 123% |
| ↑ | Q9BXB9 | LIM mineralization protein 2 | 14% | 60% | 50% | 123% |
| ↑ | Q6P181 | Hepatitis B virus receptor binding protein | 14% | 60% | 42% | 123% |
| ↑ | O95204 | Metalloprotease 1 | 14% | 60% | 42% | 123% |
| ↑ | Q7Z5K5 | Hypothetical protein | 14% | 60% | 25% | 123% |
| ↓ | P02766 | Transthyretin precursor | 71% | 0% | 50% | 200% |
| ↓ | P01764 | Ig heavy chain V-III region VH26 precursor | 43% | 0% | 42% | 200% |
| ↓ | P12814 | Alpha-actinin 1 | 43% | 0% | 8% | 200% |
| ↓ | Q96RU2 | Ubiquitin carboxyl-terminal hydrolase 28 | 43% | 0% | 17% | 200% |
| ↓ | Q9NYQ6 | Cadherin EGF LAG seven-pass G-type | 43% | 0% | 33% | 200% |
| | | receptor 1 precursor | | | | |
| ↓ | Q99996 | A-kinase anchor protein 9 | 43% | 0% | 0% | 200% |
| ↓ | Q7Z3L8 | Hypothetical protein DKFZp686P21111 | 43% | 0% | 25% | 200% |
| ↓ | Q9NPP6 | Immunoglobulin heavy chain variant | 43% | 0% | 33% | 200% |
| ↓ | P10720 | Platelet factor 4 variant precursor | 86% | 20% | 58% | 124% |
| ↓ | P02776 | Platelet factor 4 precursor | 71% | 20% | 33% | 113% |
| ↓ | Q15643 | Thyroid receptor interacting protein 11 | 71% | 20% | 33% | 113% |

### Low Molecular Weight (LMW) Serum Fraction by Disease Group

LMW sera from Normal, MCI and mild AD subjects (n=14-15 per group) were investigated using LC-MS/MS. Compared to unfractionated serum analysis, distinctive protein functional groups were identified (FIG. 2; Tables 4-6). The LMW enrichment yielded a mixture of peptides and proteins undetectable in unfractionated serum. However, “lipid transporter activity” proteins were present in both “LMW fraction by disease group” and unfractionated serum analysis for samples from normal and MCI subjects versus mild AD participants.

TABLE 4
LMW serum proteins with a significantly different spectral count in
mild AD versus Normal patients. The Normal (n = 14), MCI
(n = 14) and mild AD (n = 15) samples were pooled
prior to LC-MC/MS.

| | | | Spectra | | | % difference |
|---|---|---|---|---|---|---|
| Regulation | Accession | Protein Name | Normal | MCI | mild AD | mild AD/Normal |
| ↑ | P13645 | Keratin, type I cytoskeletal 10 | 0 | 57 | 4 | 200% |
| ↑ | P35908 | Keratin, type II cytoskeletal 2 epidermal | 0 | 16 | 4 | 200% |
| ↑ | P02671 | Fibrinogen alpha chain precursor | 1 | 5 | 7 | 150% |
| ↑ | Q6PYX1 | Hepatitis B virus receptor binding protein | 1 | 8 | 5 | 133% |
| ↑ | O75179 | KIAA0697 protein | 1 | 2 | 5 | 133% |
| ↑ | P55056 | Apolipoprotein C-IV precursor | 2 | 2 | 7 | 111% |
| ↓ | Q6GTG1 | Vitamin D-binding protein | 6 | 6 | 0 | 200% |
| ↓ | O95978 | VH1 protein precursor | 6 | 1 | 0 | 200% |
| ↓ | P01781 | Ig heavy chain V-III region GAL | 5 | 4 | 0 | 200% |
| ↓ | P01614 | Ig kappa chain V-II region Cum | 4 | 2 | 0 | 200% |
| ↓ | P13798 | Acylamino-acid-releasing enzyme | 4 | 0 | 0 | 200% |
| ↓ | P55073 | Type III iodothyronine deiodinase | 4 | 0 | 0 | 200% |
| ↓ | Q6MZW0 | Hypothetical protein DKFZp686J11235 | 6 | 5 | 1 | 143% |
| ↓ | Q5CZ94 | Hypothetical protein DKFZp781M0386 | 8 | 6 | 2 | 120% |
| ↓ | P01011 | Alpha-1-antichymotrypsin precursor | 12 | 13 | 4 | 100.00% |

TABLE 5
LMW serum proteins with a significantly different spectral count
in mild AD versus MCI patients. The Normal (n = 14), MCI
(n = 14) and mild AD (n = 15) samples were pooled prior
to LC-MC/MS.

| | | | Spectra | | | % difference |
|---|---|---|---|---|---|---|
| Regulation | Accession | Protein Name | Normal | MCI | mild AD | mild AD/Normal |
| ↑ | P02654 | Apolipoprotein C-1 precursor | 6 | 2 | 9 | 127% |
| ↑ | P55056 | Apolipoprotein C-IV precursor | 2 | 2 | 7 | 111% |
| ↓ | Q6GTG1 | Vitamin D-binding protein | 6 | 6 | 0 | 200% |
| ↓ | P01824 | Ig heavy chain V-II region WAH | 0 | 5 | 0 | 200% |
| ↓ | P02533 | Keratin, type I cytoskeletal 14 | 0 | 5 | 0 | 200% |
| ↓ | P15924 | Desmoplakin | 0 | 5 | 0 | 200% |
| ↓ | Q5KSL6 | Diacylglycerol kinase kappa | 0 | 5 | 0 | 200% |
| ↓ | P31151 | S100 calcium-binding protein A7 | 0 | 4 | 0 | 200% |
| ↓ | Q5T749 | Novel protein | 0 | 4 | 0 | 200% |
| ↓ | Q14664 | Keratin 10 | 1 | 4 | 0 | 200% |
| ↓ | P01781 | Ig heavy chain V-III region GAL | 5 | 4 | 0 | 200% |
| ↓ | P13645 | Keratin, type I cytoskeletal 10 | 0 | 57 | 4 | 174% |
| ↓ | Q6MZW0 | Hypothetical protein DKFZp686J11235 | 6 | 5 | 1 | 133% |
| ↓ | P04264 | Keratin, type II cytoskeletal 1 | 6 | 78 | 17 | 128% |
| ↓ | P35908 | Keratin, type II cytoskeletal 2 epidermal | 0 | 16 | 4 | 120% |
| ↓ | P05452 | Tetranectin precursor | 0 | 4 | 1 | 120% |
| ↓ | P23945 | Follicle-stimulating hormone receptor precursor | 1 | 7 | 2 | 111% |
| ↓ | P01011 | Alpha-1-antichymotrypsin precursor | 12 | 13 | 4 | 106% |
| ↓ | Q5CZ94 | Hypothetical protein DKFZp781M0386 | 8 | 6 | 2 | 100% |

TABLE 6
LMW serum proteins with a significantly different spectral count in MCI versus
Normal subjects. The samples from Normal (n = 14), MCI (n = 14)
and mild AD (n = 15) subjects were pooled for LC-MC/MS analysis.

| | | | Spectra | | | % difference |
|---|---|---|---|---|---|---|
| Regulation | Accession | Protein Name | Normal | MCI | mild AD | mild AD/Normal |
| ↑ | P13645 | Keratin, type I cytoskeletal 10 | 0 | 57 | 4 | 100% |
| ↑ | P35908 | Keratin, type II cytoskeletal 2 epidermal | 0 | 16 | 4 | 100% |
| ↑ | P01824 | Ig heavy chain V-II region WAH | 0 | 5 | 0 | 100% |
| ↑ | P02533 | Keratin, type I cytoskeletal 14 | 0 | 5 | 0 | 100% |
| ↑ | P15924 | Desmoplakin | 0 | 5 | 0 | 100% |
| ↑ | Q5KSL6 | Diacylglycerol kinase kappa | 0 | 5 | 0 | 100% |
| ↑ | P31151 | S100 calcium-binding protein A7 | 0 | 4 | 0 | 100% |
| ↑ | Q5T749 | Novel protein | 0 | 4 | 0 | 100% |
| ↑ | P05452 | Tetranectin precursor | 0 | 4 | 1 | 100% |
| ↑ | P04264 | Keratin 1 | 6 | 78 | 17 | 86% |
| ↑ | Q6PYX1 | Hepatitis B virus receptor binding protein | 1 | 8 | 5 | 78% |
| ↑ | P23945 | Follicle-stimulating hormone receptor precursor | 1 | 7 | 2 | 75% |
| ↑ | P02671 | Fibrinogen alpha chain precursor | 1 | 5 | 7 | 67% |
| ↑ | Q14664 | Keratin 10 | 1 | 4 | 0 | 60% |
| ↑ | P02753 | Plasma retinol-binding protein precursor | 4 | 14 | 7 | 56% |
| ↓ | P13798 | Acylamino-acid-releasing enzyme | 4 | 0 | 0 | 100% |
| ↓ | P55073 | Type III iodothyronine deiodinase | 4 | 0 | 0 | 100% |
| ↓ | P01771 | Ig heavy chain V-III region HIL | 4 | 0 | 2 | 100% |
| ↓ | O95978 | VH1 protein precursor | 6 | 1 | 0 | 71% |
| ↓ | P01764 | Ig heavy chain V-III region VH26 precursor | 7 | 2 | 5 | 56% |

### Low Molecular Weight (LMW) Serum Fraction by Longitudinal Disease Progression

Finally, LC-MS/MS was performed to analyze LMW serum samples from three participants before and after cognitive decline. Proteins differentially abundant between these two sample types were grouped into a “protease inhibitor activity” functional category (FIG. 2; Table 7).

TABLE 7
LMW serum proteins with a significantly different spectral count in three
same subject samples before versus after cognitive decline.

| | | | | | | | | | % difference |
|---|---|---|---|---|---|---|---|---|---|
| | | | Spectra | | | | | | after/before |
| | | | before cognitive decline | | | after cognitive decline | | | cognitive |
| Regulation | Accession | Protein Name | Sample A | Sample B | Sample C | Sample A | Sample B | Sample C | decline |
| ↑ | P78547 | Prosaposin | 0 | 0 | 0 | 1 | 2 | 1 | 200% |
| ↑ | P08493 | Matrix gla protein | 0 | 0 | 0 | 3 | 0 | 1 | 200% |
| ↑ | Q32LZ2 | Biliverdin reductase b | 0 | 0 | 0 | 1 | 0 | 3 | 200% |
| ↑ | Q86UQ9 | Citron | 0 | 0 | 0 | 2 | 2 | 0 | 200% |
| ↑ | O95740 | Serine (or cysteine) proteinase | 0 | 0 | 0 | 1 | 0 | 3 | 200% |
| | | inhibitor, clade b, member 4 | | | | | | | |
| ↑ | Q6EZF6 | Predicted: similar to neutrophil | 0 | 2 | 1 | 0 | 4 | 18 | 152% |
| | | defensin 1 precursor | | | | | | | |
| ↑ | P31151 | S100 calcium-binding protein | 0 | 1 | 0 | 1 | 1 | 4 | 143% |
| | | a7 | | | | | | | |
| ↑ | Q59EA4 | Phospholipase d1, | 0 | 0 | 1 | 2 | 1 | 2 | 133% |
| | | phophatidylcholine-specific | | | | | | | |
| ↑ | Q16782 | Serum amyloid a2 | 0 | 0 | 1 | 1 | 1 | 2 | 120% |
| ↑ | P00695 | Lysozyme precursor | 1 | 0 | 3 | 1 | 2 | 5 | 67% |
| ↑ | P02042 | Delta globin | 22 | 9 | 12 | 50 | 14 | 13 | 57% |
| ↓ | O00109 | Keratin 9 | 6 | 1 | 7 | 0 | 0 | 0 | 200% |
| ↓ | Q9P190 | Inter-alpha (globulin) inhibitor | 2 | 4 | 4 | 0 | 0 | 0 | 200% |
| | | h4 | | | | | | | |
| ↓ | Q02985 | Complement factor h isoform a | 4 | 2 | 4 | 1 | 0 | 0 | 164% |
| | | precursor | | | | | | | |
| ↓ | Q2KHQ6 | Apolipoprotein I1 isoform a | 3 | 3 | 1 | 0 | 1 | 0 | 150% |
| | | precursor | | | | | | | |
| ↓ | Q6GSJ0 | Keratin 1 | 36 | 5 | 7 | 3 | 4 | 3 | 131% |
| ↓ | O43608 | Roundabout, axon guidance | 2 | 2 | 0 | 0 | 1 | 0 | 120% |
| | | receptor, homolog 2 | | | | | | | |
| ↓ | Q9UGQ0 | Rsb-66 protein | 2 | 0 | 2 | 0 | 0 | 1 | 120% |
| ↓ | Q5SRP4 | Apolipoprotein m | 4 | 4 | 2 | 2 | 1 | 0 | 108% |
| ↓ | Q7L5M9 | Keratin 10 | 8 | 7 | 11 | 2 | 3 | 3 | 106% |
| ↓ | P05165 | Propionyl-coenzyme a | 10 | 7 | 5 | 3 | 0 | 5 | 93% |
| | | carboxylase, alpha polypeptide | | | | | | | |
| | | precursor | | | | | | | |
| ↓ | Q9UI14 | Hect domain and rid 5 | 3 | 3 | 2 | 1 | 1 | 1 | 91% |
| ↓ | Q8IYJ6 | Alpha 1b-glycoprotein | 3 | 5 | 5 | 1 | 3 | 2 | 74% |
| ↓ | P35443 | Thrombospondin 4 precursor | 2 | 1 | 1 | 1 | 1 | 0 | 67% |
| ↓ | P07360 | Complement component 8, | 25 | 36 | 64 | 23 | 24 | 16 | 66% |
| | | gamma polypeptide | | | | | | | |
| ↓ | Q4F786 | Beta globin | 289 | 100 | 110 | 109 | 61 | 95 | 61% |
| ↓ | Q9UC65 | Platelet factor 4 (chemokine (c- | 13 | 13 | 10 | 7 | 7 | 7 | 53% |
| | | x-c motif) ligand 4) | | | | | | | |

### Reverse Phase Protein Arrays

To verify the protein abundance of selected candidate biomarkers identified by mass spectrometry analysis, RPPAs were constructed using age and gender matched LMW serum samples from Normal, MCI and mild AD participants. Furthermore, LMW serum samples from two groups of MCI participants were included: group (a) that progressed into mild AD, and group (b) who remained stable at MCI over a time span of 1-2 years. For each participant, blood samples were collected at two distinct time points, thus providing before and after cognitive decline samples from the same patient. Three potential biomarker candidates were selected for verification based upon our mass spectrometry analysis: biliverdin reductase b (BLVRB), S100 calcium binding protein A7 (S100A7) and estrogen receptor alpha (ERA). To evaluate the expression of additional proteins involved in heme degradation, we investigated heme oxygenase 1 (HO1) and biliverdin reductase (BLVR). Cu/Zn superoxide dismutase (SOD), matrix metallopeptidase 9 (MMP9) and platelet-derived growth factor receptor (PDGFR Tyr716) were further included based on their biological significance and previous observations in our laboratory.

No significant difference in abundance of any of the investigated proteins was found when comparing sera from Normal, MCI and mild AD subjects before and after cognitive decline. A Spearman's Rho analysis was then used for all non-normalized intensities of the individual antibody stains to discover potential protein linkages. A ratio was calculated for each antibody pair that had met the cut-off criteria and these ratios were compared in Normal, MCI and mild AD, as well as before and after cognitive decline, samples.

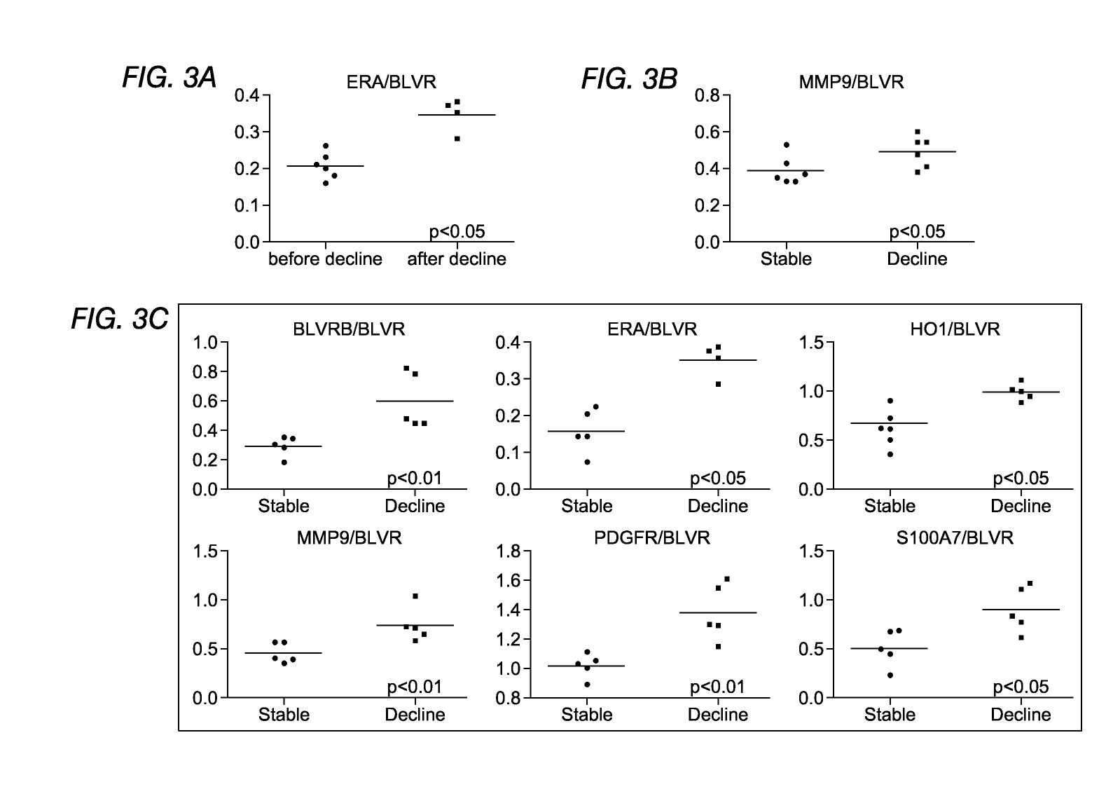

Comparing serum collection 1 (before cognitive decline) and collection 2 (after cognitive decline) in the MCI group progressing to mild AD revealed an increase in the ratio of ERA/BLVR (FIG. 3A). This same ratio was not different in the stable MCI group over the same time span. Furthermore, before progression to mild AD, declining MCI participants had an increased ratio of MMP9/BLVR compared to patients with stable MCI (FIG. 3B). After cognitive decline, six protein abundance ratios were elevated compared to the stable MCI group: BLVRB/BLVR, ERA/BLVR, HO1/BLVR, MMP9/BLVR, PDGFR Tyr716/BLVR and S100A7/BLVR (FIG. 3C).

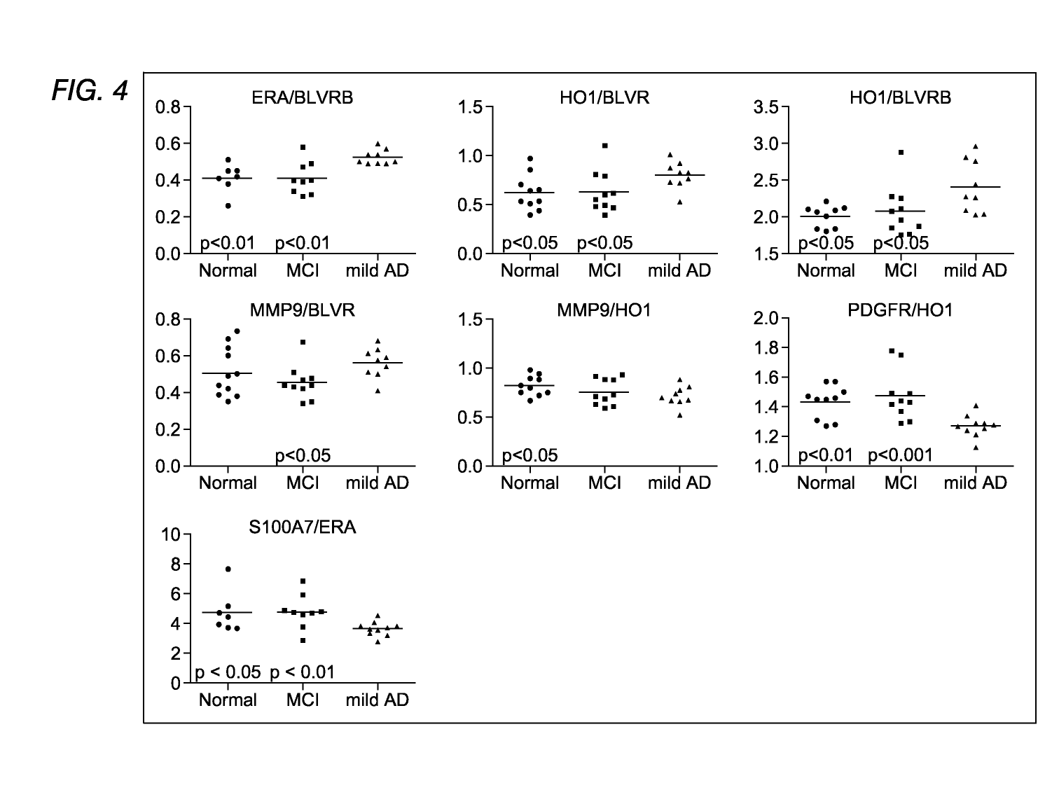

To further compare disease groups LMW serum samples from 10 age and gender matched Normal, MCI and mild AD participants were printed on RPPAs and analyzed with the same antibodies. The protein ratios of ERA/BLVRB, HO1/BLVR and HO1/BLVRB were elevated in mild AD serum versus Normal and MCI (FIG. 4). A similar ratio increase was significant only in mild AD versus MCI serum for MMP9/BLVR. For PDGFR Tyr716/HO1 and S100A7/ERA, the ratio was reduced in the serum of patients with mild AD compared to Normal subjects, as well as sera from patients with MCI. The reduction of MMP9/HO1 was significant only in mild AD versus Normal subjects.

### Discussion

One of the remaining challenges in AD is the early disease diagnosis. For successful therapy, it is necessary to begin treatment before irreversible neurodegeneration has progressed. However, currently AD is diagnosed by neuropsychological evaluation, which relies on symptoms triggered by severe neurodegeneration. Furthermore, establishing a definite diagnosis requires neuropathologic examination of postmortem brain tissue. Serum from a community-based cohort of Normal and MCI participants was studied. These subjects were followed with extensive psychometric evaluation bi-annually over a period of five years. Using stringent and generally accepted criteria (Petersen et al. (1999) Arch Neurol 56:303-308; Reisberg B (2007) Int Psychogeriatr 19:421-456) for subject classification as well as inclusion and exclusion criteria, a very well-characterized set of samples was secured. Moreover, due to the nature of this prospective study and the increased progression rate of MCI to AD (12% per year) compared to age-matched cognitively normal controls (1-2% per year) (Petersen R C J Intern Med (2004) 256:183-194), serum samples from study participants before and after the onset of dementia were able to be collected. The sample set evaluated during the course of the study proved to be very valuable for biomarker discovery study due to its exceptionally well-characterized cohort of subjects with same subject samples before and after cognitive decline.

LC-MS/MS was used to identify potential biomarker candidates. Between 468 and 2378 proteins were identified for each experimental cohort (unfractionated serum, LMW fraction, by disease group and before and after cognitive decline), of which up to 42 were selected as potential biomarkers. Classifying the candidates from unfractionated serum analysis using functional protein categories according to GO terms, “metal ion binding” and “transition metal ion binding” proteins were found to differentiate mild AD or MCI subjects from Normal subjects. The only functional protein category overlapping between the different investigative approaches (unfractionated serum versus LMW serum proteome) was that of “lipid transporter activity”.

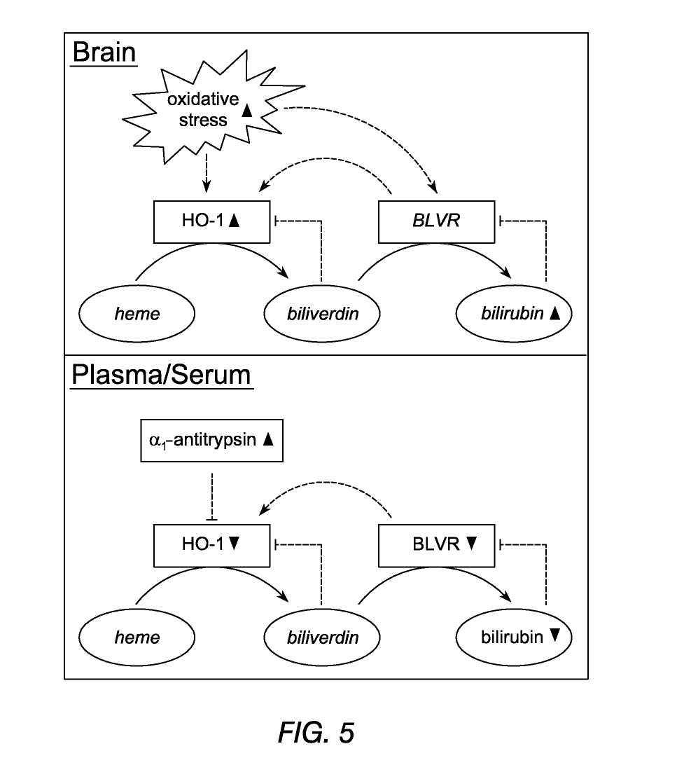

To verify selected biomarker candidates and complement the MS-based discovery phase of the project, reverse phase protein arrays (RPPA) were used. Of the candidates identified, the serum level of BLVRB, S100A7 and ERA were verified initially. BLVRB reduces biliverdin, a degradation product of heme, to bilirubin. While BLVRB is found abundantly in adult erythrocytes, BLVRA is actually the major biliverdin reductase in human adult liver (Pereira et al. Nat. Struct. Biol. (2001) 8:215-20). In fact, BLVRB shares very little sequence identity with BLVRA, but was rather found to be identical with flavin reductase (Shalloe et al. Biochem. J. (1996) 316:385-387). To extend the evaluation to the general heme degradation pathway, an antibody that recognizes both forms of biliverdin reductase (BLVR) was included as well as another against HO1, which is upregulated in AD brain but downregulated in AD plasma or serum (FIG. 5). MMP9, PDGFR Y716 and SOD were also included in the analysis.

Of the 15 protein ratios observed to be significantly altered in mild AD sera compared to Normal or MCI, 14 involved either 1-101, BLVR or BLVRB. This strongly implicates the heme degradation pathway as a potential biomarker target for AD. In all 12 protein ratios with BLVR or BLVRB as the denominator, the ratio increases, indicating a reduction in either BLVRA or BLVRB. This also is the case for the protein ratios of HO1/BLVR and HO1/BLVRB, thus pointing to a skewed ratio of heme degradation enzymes. The activity pattern of heme catabolism is markedly different between brain and periphery, with opposite extremes (upregulation in brain and downregulation in plasma or serum) clearly differentiating AD from cognitively normal subjects.

Remarkably, in the studies described herein, a significant difference (an increase in the ratio) was found between same patient samples before and after cognitive decline for the ratio of ERA/BLVR. This was shown not to have been due to an increase in age because in a similar group that remained stable at MCI over the same time span this ratio did not change.

In conclusion, using LC-MS/MS applied to MCI patient sera, several serum biomarker candidates were identified that were correlated with MCI or mild AD compared to Normals, or that changed in the same patient following cognitive decline. Many of these candidates were biologically associated with the disease process, further justifying their status as potential biomarkers. In a first verification study using RPPAs, selected candidates were quantified in Normal, MCI and mild AD serum. Differences in protein ratios were found that distinguished mild AD sera from either MCI or Normal, indicating their potential for monitoring the progression from cognitively normal or MCI status to mild AD. As over 90% of these protein ratios contained at least one enzyme involved in heme catabolism, components of the heme degradation pathway can be valuable as potential biomarkers.

Example 2 describes in greater detail some of the materials and methods used in Example 1.

### Example 2

Blood samples were collected from a community-based cohort of cognitively normal (control or Normal) and mild cognitively impaired (MCI). Subjects were recruited and followed clinically for a period of five years. Subject classification was based on extensive and repeated psychometric evaluation according to previously published criteria (Petersen R C J Intern Med (2004) 256:183-194; Petersen et al. (1999) Arch Neurol 56:303-308; Reisberg B (2007) Int Psychogeriatr 19:421-456). Diagnosis was based on bi-yearly cognitive testing, including Logical Memory I and II, Wisconsin Card Sorting Test, Trail Making Test A and B, Boston Naming Test, Draw-A Clock, Geriatric Depression Scale, Word Fluency (Phonemic and Semantic) as well as videotaped Global Clinical Dementia Rating (CDR) with informant. Cognitively normal subjects had a CDR of 0, a CDR memory component of 0 and a maximum sum of CDR boxes of 1 at baseline. MCI subjects had a CDR of 0.5 with confirmed memory complaint, abnormal memory according to age and education but no dementia, normal general cognitive function and normal daily living activities. Progression to dementia (mild AD) was determined by a sum of CDR boxes of 3.5 or more, NINCDS-ADRDA criteria and clinical judgment. Demographic data is shown in the following Tables 8 and 9.

TABLE 8
Demographic data for sample sets used in Whole Serum (A) (whole
serum MS analysis of individual patient samples of normals, MCI and
mild AD patients), LMW (B) (low molecular weight MS analysis of
pooled patient samples of normals, MCI and mild AD patients) and
Protein Array (A) (low molecular weight protein array analysis of
individual samples of normals, MCI and mild AD patients).

| | | n | Age |
|---|---|---|---|
| Experiment | Disposition | (female/male) | [years] |
| MS Whole Serum | Normal | 7 (6/1) | 69.4 ± 8.9 |
| by group | MCI | 5 (2/3) | 80.4 ± 5.3* |
| | mild AD | 12 (8/4) | 81.5 ± 3.0* |
| MS LMW | Normal | 14 (9/5) | 78.9 ± 3.9 |
| by group | MCI | 14 (9/5) | 81.5 ± 4.8 |
| | mild AD | 15 (10/5) | 80.9 ± 2.7 |
| RPPA LMW | Normal | 10 (5/5) | 78.3 ± 2.5 |
| by group | MCI | 10 (5/5) | 80.4 ± 3.8 |
| | mild AD | 10 (5/5) | 79.3 ± 3.4 |

Errors represent SD.
*p < 0.05 versus normal.

TABLE 9
Demographic data for mass spectrometry (MS) and reverse phase
protein array (RPPA) low molecular weight (LMW) serum analysis of
same patient serum samples before and after significant cognitive
decline.

| | | | n | | |
|---|---|---|---|---|---|
| | | Sampling | (female/ | Age | ΔTime |
| Experiment | Development | date | male) | [years] | [years] |
| MS LMW | decline | a | 3 (2/1) | 79.0 ± 2.7 | |
| longitudinal | | b | 3 (2/1) | 80.0 ± 2.7 | 1 ± 0.0 |
| RPPA LMW | stable | a | 6 (4/2) | 80.5 ± 5.6 | |
| longitudinal | | b | 6 (4/2) | 81.8 ± 5.7 | 1.5 ± 0.5 |
| | decline | a | 6 (4/2) | 80.2 ± 7.2 | |
| | | b | 6 (4/2) | 82.0 ± 6.9 | 1.8 ± 0.4 |

Patients marked as “stable” did not change cognitively over the same time span (MCI => MCI).
Errors represent SD.

### Serum Sample Collection

Consecutive blood samples were collected about every six months from a cohort of cognitively normal controls as well as MCI and mild AD subjects that were followed clinically for five years. Blood collection tubes containing no anticoagulant were stored at 4° C. overnight to allow the blood to clot. Subsequently, the serum was separated by centrifugation at 1800 g and 4° C. for 10 min. Following separation, the serum was mixed by gently inverting it in a 15 ml Falcon tube and aliquots of 500 μl were frozen at −80° C. until analysis.

### Low Molecular Weight Fractionation

Serum samples were prepared in a loading solution with 25 μl of serum, 75 μl 2×SDS Tris Glycine Sample buffer, 15 μl 1M DTT, and 3 μl Bromophenol Blue. A Mini Prep Cell Apparatus (Bio-Rad) was used according to manufacturer specifications to isolate low molecular weight proteins. A 4% stacking and 10% cylindrical gel were used for electrophoretic separation, followed by elution with a peristaltic pump into five—500 μL aliquots. Fractions containing proteins and peptides with molecular weights <30 kDa were combined and concentrated with Microcon Ultracel YM-3 (Millipore) filter cartridges according to manufacturer specifications. A final volume of 40 μL was achieved by adding 1× Tris-Glycine SDS Running Buffer. For reverse phase protein microarray printing, samples were diluted 1:2 in a solution of 2× Tris-Glycine SDS Sample Buffer with 20% glycerol and 2.5% 2-mercaptoethanol. For mass spectrometry analysis, SDS was removed from the LMW fraction by tricholoroacetic acid (TCA) precipitation. Samples were incubated with an equal volume of 10% TCA (w/v) on ice for 1 hour and then centrifuged at 15,000 g and 4° C. for 30 minutes. The pellet containing the precipitated proteins/peptides was washed in cold acetone and dissolved in 8 M urea.

### Mass Spectrometry

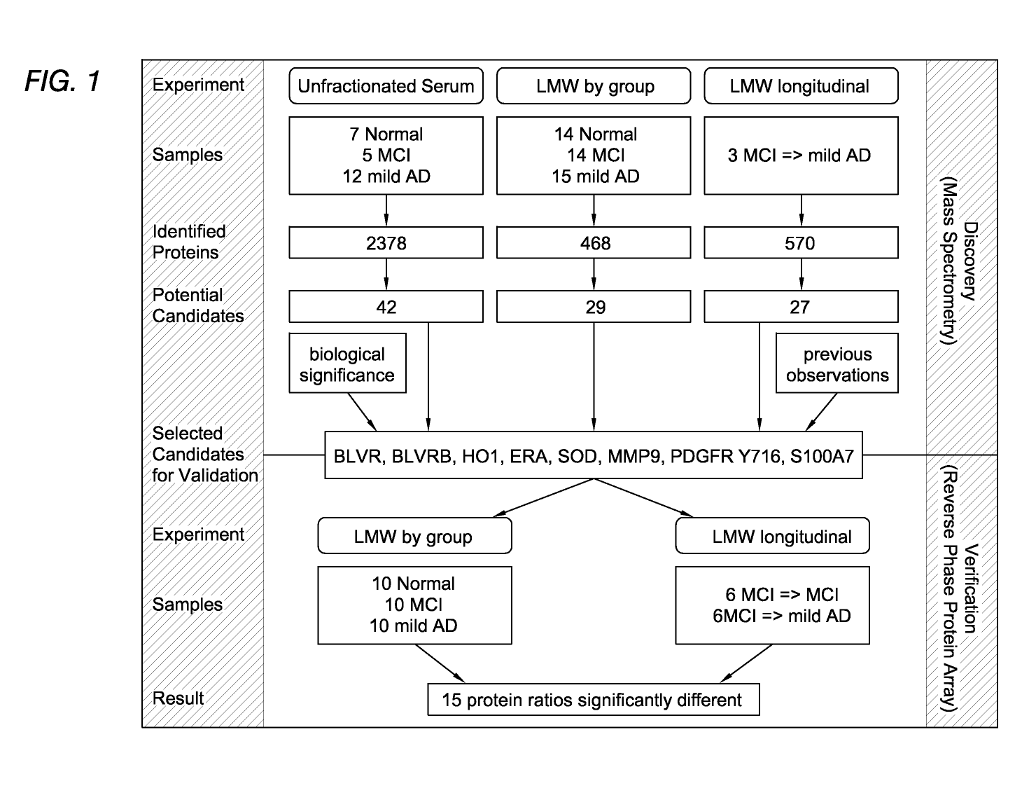

LC-MS/MS analysis was performed using a Thermo hybrid LTQ-Orbitrap mass spectrometer. Serum samples were studied either as trypsin digested unfractionated serum or low molecular weight (LMW) serum fractions. LMW fractions were each reduced and alkylated by reaction with 15 mM DTT and 50 mM iodoacetamide respectively. Study samples were divided into the following three sets (FIG. 1): (A) Unfractionated Serum: serum samples from individual Normal (n=7), MCI (n=5) and mild AD (n=12) subjects; (B) LMW fraction by disease group: LMW fractions of three pooled samples consisting of serum from 14 Normal, 14 MCI and 15 mild AD subjects respectively; (C) LMW fraction longitudinal by disease progression: LMW fractions of serum samples from three individual patients collected before and after significant cognitive decline.

Peptide and protein identification was performed using the SEQUEST algorithm to search the MS data against the human protein database available at NCBI. To obtain high confidence identifications the search results were filtered based on rank of match (RSp=1), cross-correlation score for the peptide molecular ion charge state (XCorr>1.9 (1+), 2.2 (2+) and 3.5 (3+)), difference between the first and second ranked match (ΔCn>0.1) and the probability of a random match (p<0.01). Raw data were visualized, filtered, sorted and manually confirmed using Scaffold (Proteome Software Inc.). Candidates for further analysis were selected by filtering according to the following criteria: (A) Unfractionated serum: a probability score of less than 1.00E-03, identification in at least 33% of samples belonging to one disease group and showing a greater than 50% difference between compared groups; (B) LMW fraction by disease group: identification of at least four individual spectra corresponding to the protein in a single disease group and showing a greater than 50% difference between compared groups; (C) LMW fraction by longitudinal disease progression: identification of at least four individual spectra corresponding to this protein in a single disease group, showing a greater than 50% difference between compared groups and the direction of change in spectral count between before and after cognitive decline had to be the same in at least two subjects and could not be counter directional in the third subject.

Analysis according to biological significance was performed using Ingenuity Pathway Analysis (Ingenuity Systems), DAVID bioinformatics database (National Institute of Allergy and Infectious Diseases, NIH), GeneCards (Weizmann Institute of Science and Xennex), GNF SymAtlas (Genomics Institute of the Novartis Research Foundation) and a custom software program developed in-house that allows batch searching of Medline through PubMed using automatically combined lists of proteins and specified search terms.

### Reverse-Phase Protein Arrays

Samples were denatured by heating at 100° C. for 7 minutes. Two-fold dilution curves (neat, 1:2, 1:4, 1:8) were printed in an array format onto FAST nitrocellulose slides (Whatman) with an Aushon 2470 arrayer equipped with 350 μm solid pins. Humidity was set to 50% producing a final spot diameter of 650 μm. Arrays were stored with dessicant at −20° C. prior to immunostaining.

Arrays were blocked (1-Block, Applied Biosystems) for 1 hour and subsequently probed with antibodies, previously validated by immunoblotting, to Biliverdin Reductase B (BVRB) (Abnova), Biliverdin Reductase (Stressgen), Cu/Zn Superoxide dismutase (Stressgen), Estrogen receptor alpha (Cell Signaling), Heme Oxygenase-1 (BIOMOL International, LP), Matrix Metalloproteinase-9 (BIOMOL International, LP), PDGFR Tyr716 (Upstate), S100A7 (Abnova) and Beta Globin (Abnova). Immunostaining was performed on an automated slide stainer per manufacturer's instructions (Autostainer CSA kit, Dako). Each slide was incubated with a single primary antibody at room temperature for 30 minutes. The negative control slide was incubated with antibody diluent. Secondary antibody was goat anti-rabbit IgG H+L (1:5000) (Vector Labs, Burlingame, Calif.) or rabbit anti-mouse IgG (1:10) (Dako). Subsequent protein detection was amplified via horseradish peroxidase mediated biotinyl tyramide with chromogenic detection (Diaminobenzidine) per manufacturer's instructions (Dako). Arrays were also stained for total protein using SYPRO Ruby (Invitrogen Corporation) and visualized on a NovaRay (Alpha Innotech).

Each antibody array was scanned on a flatbed scanner (UMAX PowerLook 1120), spot intensity analyzed, and a standardized, single data value was generated for each sample on the array (ImageQuant 5.2, Molecular Dynamics).

Staining intensities were normalized to Beta Globin because of its molecular weight of 16 kDa, which is within the molecular weight range of the LMW serum fraction, thus ensuring inclusion of the full-length protein. Furthermore, mass spectrometry data indicated that Beta Globin is equally abundant between Normal, MCI and mild AD LMW serum samples (data not shown).

A Spearman's Rho non-parametric analysis of the non-normalized spot intensities was used to identify potential protein linkages. Correlations with a Spearman's Rho coefficient ≧0.85 and a p value ≦0.05 were considered for further analysis.

### Additional Software

Statistical analysis was performed using the SPSS 16 software package. Unless otherwise specified, a p value of <0.05 was used to indicate statistical significance. Proteins and peptides identified by MS were grouped by protein functional category.

While this invention has been described in connection with certain embodiments, it will be appreciated by those skilled in the art that various modifications and changes may be made without departing from the scope of the present disclosure. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. Thus, while the present disclosure has described certain exemplary embodiments, it is to be understood that the disclosure is not limited to the disclosed embodiments, but, on the contrary, is intended to cover various modifications and equivalent arrangements included within the spirit and scope of the appended claims, and equivalents thereof.

1. A method for diagnosing Alzheimer's Disease (AD) in a subject, comprising:
obtaining a biological sample from a subject suspected of being at risk for said AD;
determining a level of expression of at least one first biomarker in said biological sample from said subject, wherein said first biomarker is selected from the group consisting of biliverdin reductase (BLVR), biliverdin reductase B (BLVRB), estrogen receptor alpha (ERA), S100A7, hemeoxygenase 1 (HO1), matrix metalloproteinase 9 (MMP9) and platelet derived growth factor receptor beta (PDGFR);
determining a level of expression of at least one second biomarker in said biological sample from said subject, said second biomarker being different from said first biomarker and being selected from the group consisting of BLVR, BLVRB, ERA, S100A7, HO1, MMP9 and PDGFR;
determining a ratio of said first biomarker to said second biomarker; and
comparing the level of the ratio to a predetermined level indicative of a subject not having AD, wherein a significant difference in said ratio compared to the predetermined level indicates a greater likelihood of AD in the subject.

2-29. (canceled)

30. The method of claim 1, wherein said first biomarker comprises ERA and said second biomarker comprises BLVRB.

31. The method of claim 1, wherein said first biomarker comprises MMP9 and said second biomarker comprises BLVR.

32. The method of claim 1, wherein said first biomarker comprises S100A7 and said second biomarker comprises ERA.

33. The method of claim 1, wherein said first biomarker comprises HO1 and said second biomarker comprises BLVR.

34. The method of claim 1, wherein said first biomarker comprises MMP9 and said second biomarker comprises HO1.

35. The method of claim 1, wherein said first biomarker comprises HO1 and said second biomarker comprises BLVRB.

36. The method of claim 1, wherein said first biomarker comprises PDGFR and said second biomarker comprises HO1.

37. The method of claim 1, wherein the second biomarker is selected from the group consisting of BLVR, BLVRB, ERA, S100A7, and PDGFR

38. The method of claim 1, wherein said biological sample is blood, serum or plasma.

39. The method of claim 1, wherein determining the level of expression of the first and second biomarkers comprises determining the level of mRNA for the first and second biomarkers.

40. The method of claim 1, wherein determining the level of expression of the first and second biomarkers comprises determining the level of protein for the first and second biomarkers.

41. The method of claim 40, wherein determining the level of expression of the first and second biomarkers comprises contacting said biological sample with antibodies against the first and second biomarkers.

42. The method of claim 41, wherein determining the level of expression of the first and second biomarkers comprises an assay selected from the group consisting of immunoassay, mass spectrometry, immuno-mass spectrometry and suspension bead array.

43. The method of claim 42, wherein said immunoassay is an enzyme linked immunosorbent assay (ELISA).

44. The method of claim 42, wherein said mass spectrometry comprises tandem mass spectroscopy (MSMS).

45. A method for monitoring the progress of a neurological condition selected from the group consisting of AD and mild cognitive impairment (MCI) in a subject, comprising:
obtaining a first biological sample from a subject with said neurological condition at a first time;
obtaining a second biological sample from said subject at a second time;
determining a level of expression of at least one first biomarker in said first biological sample and said second biological sample, said first biomarker being selected from the group consisting of BLVR, BLVRB, ERA, S100A7, HO1, MMP9 and PDGFR;
determining a level of expression of at least one second biomarker in said first biological sample and said second biological sample, said second biomarker being different from said first biomarker and being selected from the group consisting of BLVR, BLVRB, ERA, S100A7, HO1, MMP9 and PDGFR;
determining a first ratio of said first biomarker to said second biomarker in said first biological sample;
determining a second ratio of said first biomarker to said second biomarker in said second biological sample;
comparing the level of the first ratio and the second ratio, thereby monitoring the progress of said neurological condition in said subject wherein a difference in said first ratio compared to said second ratio indicates the progress of said neurological condition.

46. The method of claim 45, wherein said first biomarker comprises ERA and said second biomarker comprises BLVR, wherein said first biomarker comprises MMP9 and said second biomarker comprises BLVR, wherein said first biomarker comprises BLVRB and said second biomarker comprises BLVR, wherein said first biomarker comprises ERA and said second biomarker comprises BLVR, wherein said first biomarker comprises HO1 and said second biomarker comprises BLVR, wherein said first biomarker comprises MMP9 and said second biomarker comprises BLVR, wherein said first biomarker comprises PDGFR and said second biomarker comprises BLVR, or wherein said first biomarker comprises S100A7 and said second biomarker comprises BLVR.

47. The method of claim 45, wherein the second biomarker is selected from the group consisting of BLVR, BLVRB, ERA, S100A7, and PDGFR.

48. The method of claim 45, wherein said biological sample is blood, serum or plasma.

49. The method of claim 45, wherein determining the level of expression of the first and second biomarkers comprises determining the level of mRNA for the first and second biomarkers.

50. The method of claim 45, wherein determining the level of expression of the first and second biomarkers comprises determining the level of protein for the first and second biomarkers.

51. The method of claim 50, wherein determining the level of expression of the first and second biomarkers comprises contacting said biological sample with antibodies against the first and second biomarkers.

52. The method of claim 51, wherein determining the level of expression of the first and second biomarkers comprises an assay selected from the group consisting of immunoassay, mass spectrometry, immuno-mass spectrometry and suspension bead array.

53. The method of claim 52, wherein said immunoassay is an enzyme linked immunosorbent assay (ELISA).

54. The method of claim 52, wherein said mass spectrometry comprises tandem mass spectroscopy (MSMS).

55. A kit for detecting presence or progression of a neurological disorder, said kit comprising:
a first agent that specifically detects at least one first biomarker selected from the group consisting of BLVR, BLVRB, ERA, S100A7, HO1, MMP9, and PDGFR;
a second agent that specifically detects at least one second biomarker different from the first biomarker and selected from the group consisting of BLVR, BLVRB, ERA, S100A7, HO1, MMP9, and PDGFR; and
instructions for using the kit components to determine the level of expression of said first biomarker and said second biomarker and to determine a ratio of said first biomarker to said second biomarker in a person at risk for said neurological condition.

56. The kit of claim 55, wherein said first agent that specifically detects said first biomarker is an antibody that binds to said first biomarker.

57. The kit of claim 55, wherein said second agent that specifically detects said second biomarker is an antibody that binds to said second biomarker.
